(19) United States
(12) Patent Application Publication
(10) Pub. No.: US 2009/0143440 A1
(43) Pub. Date: Jun. 4, 2009

(54) Pyrazoles Useful in the Treatment of Inflammation

(75) Inventors: Benjamin Pelcman, Solna (SE); Andrei Sanin, Solna (SE); Peter Nilsson, Solna (SE); Thomas Groth, Copenhagen (DK); Hasse Kromann, Copenhagen (DK)

Correspondence Address: MORGAN LEWIS & BOCKIUS LLP, 1111 PENNSYLVANIA AVENUE NW, WASHINGTON, DC, 20004, US

(73) Assignee: BIOLIPOX AB, Solna (SE)

(21) Appl. No.: 12/084,331

(22) PCT Filed: Oct. 27, 2006
(86) PCT No.: PCT/GB2006/004009

§ 371 Date: Oct. 1, 2008

Related U.S. Application Data
(60) Provisional application No. 60/731,482, filed on Oct. 31, 2005.

(51) Int. Cl. A61K 31/4439; C07D 403/12; A61P 25/12

(52) U.S. Cl. 514/341; 546/275.4

(57) ABSTRACT

There is provided compounds of formula (I), wherein R¹, R², X¹, X² and n have meanings given in the description, and pharmaceutically-acceptable salts thereof, which compounds are useful in the treatment of diseases in which inhibition of the activity of a lipoxygenase (e.g. 15-lipoxygenase) is desired and/or required, and particularly in the treatment of inflammation.

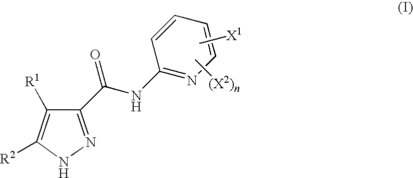

### FIELD OF THE INVENTION

The invention relates to novel pharmaceutically-useful compounds. The invention further relates to compounds that are useful in the inhibition of the activity of 15-lipoxygenase and thus in the treatment of inflammatory diseases and of inflammation generally. The invention also relates to the use of such compounds as medicaments, to pharmaceutical compositions containing them, and to synthetic routes for their production.

### BACKGROUND OF THE INVENTION

There are many diseases/disorders that are inflammatory in their nature. One of the major problems associated with existing treatments of inflammatory conditions is a lack of efficacy and/or the prevalence of side effects (real or perceived).

Asthma is a chronic inflammatory disease affecting of 6% to 8% of the adult population of the industrialized world. In children, the incidence is even higher, being close to 10% in most countries. Asthma is the most common cause of hospitalization for children under the age of fifteen.

Treatment regimens for asthma are based on the severity of the condition. Mild cases are either untreated or are only treated with inhaled β-agonists. Patients with more severe asthma are typically treated with anti-inflammatory compounds on a regular basis.

There is a considerable under-treatment of asthma, which is due at least in part to perceived risks with existing maintenance therapy (mainly inhaled corticosteroids). These include risks of growth retardation in children and loss of bone mineral density, resulting in unnecessary morbidity and mortality. As an alternative to steroids, leukotriene receptor antagonists (LTRas) have been developed. These drugs may be given orally, but are considerably less efficacious than inhaled steroids and usually do not control airway inflammation satisfactorily.

This combination of factors has led to at least 50% of all asthma patients being inadequately treated.

A similar pattern of under-treatment exists in relation to allergic disorders, where drugs are available to treat a number of common conditions but are underused in view of apparent side effects. Rhinitis, conjunctivitis and dermatitis may have an allergic component, but may also arise in the absence of underlying allergy. Indeed, non-allergic conditions of this class are in many cases more difficult to treat.

Chronic obstructive pulmonary disease (COPD) is a common disease affecting 6% to 8% of the world population. The disease is potentially lethal, and the morbidity and mortality from the condition is considerable. At present, there is no known pharmacological treatment capable of changing the course of COPD.

Other inflammatory disorders which may be mentioned include:
(a) pulmonary fibrosis (this is less common than COPD, but is a serious disorder with a very bad prognosis. No curative treatment exists);
(b) inflammatory bowel disease (a group of disorders with a high morbidity rate. Today only symptomatic treatment of such disorders is available); and
(c) rheumatoid arthritis and osteoarthritis (common disabling inflammatory disorders of the joints. There are currently no curative, and only moderately effective symptomatic, treatments available for the management of such conditions).

Inflammation is also a common cause of pain. Inflammatory pain may arise for numerous reasons, such as infection, surgery or other trauma. Moreover, several malignancies are known to have inflammatory components adding to the symptomatology of the patients.

Thus, a new and/or alternative anti-inflammatory treatment would be of benefit to all of the above-mentioned patient groups. In particular, there is a real and substantial unmet clinical need for an effective antiinflammatory drug capable of treating inflammatory disorders, such as asthma, with no real or perceived side effects.

The mammalian lipoxygenases are a family of structurally-related enzymes, which catalyze the oxygenation of arachidonic acid. Three types of human lipoxygenases are known, which catalyze the insertion of molecular oxygen into arachidonic acid at carbon positions 5, 12 and 15. The enzymes are thus named 5-, 12- and 15-lipoxygenase, respectively.

Arachidonic acid metabolites that are formed following the action of lipoxygenases are known to have pronounced pathophysiological activity including pro-inflammatory effects.

For example, the primary product of the action of 5-lipoxygenase on arachidonic acid is further converted by a number of enzymes to a variety of physiologically and pathophysiologically important metabolites. The most important of these, the leukotrienes, are strong bronchoconstrictors. Huge efforts have been devoted towards the development of drugs that inhibit the action of these metabolites as well as the biological processes that form them. Drugs that have been developed to this end include 5-lipoxygenase inhibitors, inhibitors of FLAP (Five Lipoxygenase Activating Protein) and, as mentioned previously, leukotriene receptor antagonists (LTRas).

Another class of enzymes that metabolize arachidonic acid are the cyclooxygenases. Arachidonic acid metabolites that are produced by this process include prostaglandins, thromboxanes and prostacyclin, all of which possess physiological or pathophysiological activity. In particular, the prostaglandin PGE₂ is a strong pro-inflammatory mediator, which also induces fever and pain. Consequently, a number of drugs have been developed to inhibit the formation of PGE₂, including “NSAIDs” (non-steroidal antiinflammatory drugs) and “coxibs” (selective cyclooxygenase-2 inhibitors). These classes of compounds act predominantly by way of inhibition of one or several cyclooxygenases.

Thus, in general, agents that are capable of blocking the formation of arachidonic acid metabolites are likely to be of benefit in the treatment of inflammation.

### PRIOR ART

Certain pyrazole compounds that are structurally related to those described herein are commercially available. However, to the knowledge of the applicant, these compounds have never been disclosed in any printed publication and as such have no perceived utility ascribed to them.

Pyrazole-based compounds have been disclosed in several publications. For example, international patent application WO 01/57024 discloses various pyrazoles that are useful in blocking voltage-dependent sodium channels; international patent applications WO 03/020217 and WO 01/58869, and US Patent No. 2004/0192667 disclose various nitrogen-containing heterocycles, including pyrazoles, that are useful as modulators of cannabinoid receptors; international patent application WO 99/20294 discloses pyrazoles that are useful in the treatment of cystic fibrosis; international patent application WO 2005/007625 discloses anti-tuberculosis compounds that include pyrazoles; US patent No. 2002/0091116 and international patent applications WO 01/19798, WO 99/32454 and WO 2004/055815 disclose inter alia pyrazoles that may be useful as Factor Xa inhibitors; international patent application WO 2004/039795 discloses various heterocycles, including pyrazoles, for use as apolipoprotein B secretion inhibitors (and therefore useful in the treatment of inter alia hyperlipemia); international patent application WO 2002/070483 discloses various pyrazoles for use as pest control agents, international patent applications WO 2004/094380 and WO 03/084949 disclose various compounds, including pyrazoles, for use as inter alia 5-HT 1F agonists and international patent applications WO 01/21160 and WO 2004/071426 discloses antiviral compounds that include pyrazoles. There is no disclosure in any of these documents of 1(N)-unsubstituted-3-amidopyrazoles for use in treating inflammation and/or as inhibitors of lipoxygenases.

International patent application WO 2004/041789 discloses various compounds that may be useful as protein kinase inhibitors (and therefore useful in the treatment of inter alia autoimmune diseases). However, there is no specific disclosure of a 3-amidopyrazole in this document.

International patent application WO 2004/096795 discloses various heterocycles, including pyrazoles, as inhibitors of protein tyrosine kinases, international patent application WO 02/092573 discloses various heterocycles for use as inhibitors of inter alia JNK3 protein kinases and international patent application WO 01/55115 discloses various aromatic amides that may be useful as activators of caspases and inducers of apoptosis. Accordingly, the compounds disclosed in these documents may be useful in the treatment of inter alia cancer. There is no disclosure or suggestion in any of these documents of the use of such compounds as inhibitors of lipoxygenases.

International patent application WO 97/19062 discloses various pyrazoles for the treatment of skin related diseases and further mentions the use of such compounds in the treatment of various inflammatory diseases. However, this document does not mention or suggest 3-amido pyrazoles that are not substituted at the 4- and/or 5-position of the pyrazole ring with a group not containing an oxygen atom.

International patent application WO 2005/016877 discloses pyrazoles that may be useful in the inhibition of 11β-hydroxysteroid dehydrogenase-1 (and therefore useful in the treatment of inter alia diabetes). Vertuani et al., Journal of Pharmaceutical Sciences, Vol. 74, No. 9 (1985) discloses various pyrazoles that possess anti-inflammatory and analgesic activities. There is no mention or suggestion in these document of pyrazoles that are substituted in the 3-position with an heteroaromatic amido group.

Certain pyrazolecarboxylic acid hydrazides, structurally unrelated to the compounds described herein, have been disclosed as anti-inflammatory agents in Tihanyi et al, Eur. J. Med. Chem.—Chim. Ther., 1984, 19, 433 and Goel et al, J. Chem. Inf. Comput. Sci. 1995, 35, 510.

International patent application WO 03/037274 discloses various pyrazoles that may be useful in treating inflammatory pain, which mechanism works by blocking sodium channels. This document relates primarily to pyrazoles that are 1(N)-substituted and also to pyrazoles that are substituted by an amido group in the 4-position.

International patent application WO 03/068767 also relates to inter alia pyrazole-containing compounds that may be useful in treating inflammatory pain by opening potassium ion channels. However, this document relates specifically to pyrimidinyl amido compounds.

International patent application WO 96/18617 discloses numerous compounds that may be useful as inhibitors of nitric oxide synthase and therefore in the treatment of diseases such as inflammation, WO 2005/009954 and WO 2005/009539 disclose various compounds including pyrazoles for use in the inhibition of the production of inter alia IL-2, and thus in the treatment of inflammation. This document does not mention or suggest the use of the compounds disclosed therein as inhibitors of lipoxygenases. Further there is no specific disclosure in these documents of pyrazole-3-carboxylic acid amides.

International patent applications WO 2004/080999 and WO 2006/032852 both disclose various 3-amidopyrazoles for use in the treatment of inflammation. However, there is no disclosure or suggestion in any of these documents of N-unsubstituted 3-amidopyrazoles for use in such treatment.

International patent application WO 2006/032851 discloses various 3-amidopyrazoles for use in the treatment of inflammation in which the amido group is substituted with a tricyclic heterocyclic group. However, there is no disclosure or suggestion of corresponding 3-amidopyrazoles in which the amido group is substituted by a monocyclic aromatic group.

### DISCLOSURE OF THE INVENTION

According to the invention there is provided a compound of formula I,

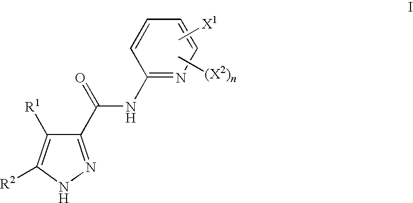

wherein
R¹ and R² independently represent H, halo or C₁₋₆ alkyl optionally substituted by one or more halo atoms;
X¹ represents H, halo or R^{3a};
X² represents:

### 1) G¹;

2) aryl or heteroaryl, both of which are optionally substituted by one or more substituents selected from A¹, —N₃, —NO₂ and —S(O)ₚR^{6e}; and
3) heterocycloalkyl, which is optionally substituted by one or more substituents selected from A², —N₃, —NO₂ and ═O;
G¹ represents halo, —R^{3a}, —CN, —C(O)R^{3b}, —C(O)OR^{3c}, —C(O)N(R^{4a})R^{5a}, —N(R^{4b})R^{5b}, —N(R^{3d})C(O)R^{4c}, —N(R^{3e})C(O)N(R^{4d})R^{5d}, —N(R^{3f})C(O)OR^{4e}, —N₃, —NO₂, —N(R^{3g})S(O)₂N(R^{4f})R^{5f}, —OR^{3h}, —OC(O)N(R^{4g})R^{5g}, —OS(O)₂R³ⁱ, —S(O)ₘR^{3j}, —N(R^{3k})S(O)₂R^{3m}, —OC(O)R³ⁿ, —OC(O)OR^{3p}, —S(O)₂N(R^{4h})R^{5h}, —S(O)₂OH, —P(O)(OR⁴ⁱ)(OR⁵ⁱ) or —C(O)N(R^{3q})S(O)₂R^{3r};
R^{3a} represents, on each occasion when used herein, C₁₋₆ alkyl optionally substituted by one or more substituents selected from Z, F, Cl, —N(R^{6b})R^{6c}, —N₃, ═O and —OR^{6d};
R^{3b}, R^{3c}, R^{3h}, R³ⁿ, R^{4a} to R^{4h}, independently represent H, Z or C₁₋₆ alkyl optionally substituted by one or more halo atoms or —OR^{6d};
R^{3d} to R^{3g}, R^{3k}, R^{3q}, R^{5a}, R^{5b}, R^{5d} and R^{5f} to R^{5h} independently represent H or C₁₋₆ alkyl optionally substituted by one or more halo atoms or —OR^{6d}; or
any of the pairs R^{4a} and R^{5a}, R^{4b} and R^{5b}, R^{4d} and R^{5d}, R^{4f} and R^{5f}, R^{4g} and R^{5g}, and R^{4h} and R^{5h}, may be linked together to form a 3- to 6-membered ring, which ring optionally contains a further heteroatom (such as nitrogen or oxygen) in addition to the nitrogen atom to which these substituents are necessarily attached, and which ring is optionally substituted by ═O or C₁₋₆ alkyl optionally substituted by one or more fluoro atoms;
R³ⁱ, R^{3j}, R^{3m}, R^{3p} and R^{3r} independently represent Z or C₁₋₆ alkyl optionally substituted by one or more substituents selected from B¹;
R⁴ⁱ and R⁵ⁱ independently represent H or C₁₋₆ alkyl optionally substituted by one or more substituents selected from B²;
Z represents, on each occasion when mentioned herein:
a) heterocycloalkyl optionally substituted by one or more substituents selected from A³ and ═O;
b) aryl or heteroaryl both of which are optionally substituted by one or more substituents selected from A⁴, —N₃, —NO₂ and —S(O)_{q}R^{7e};
A¹, A², A³ and A⁴ independently represent halo, —R^{6a}, —CN, —N(R^{6b})R^{6c} or —OR^{6d};
R^{6b} to R^{6d} independently represent, on each occasion when mentioned herein, H or C₁₋₆ alkyl optionally substituted by one or more substituents selected from B³;
R^{6a}, R^{6e} and R^{7e} independently represent C₁₋₆ alkyl optionally substituted by one or more substituents selected from B⁴; or
R^{6b} and R^{6c} may be linked together to form a 3- to 6-membered ring, which ring optionally contains a further heteroatom (such as nitrogen or oxygen) in addition to the nitrogen atom to which these substituents are necessarily attached, and which ring is optionally substituted by ═O or C₁₋₆ alkyl optionally substituted by one or more fluoro atoms;
B¹, B², B³ and B⁴ independently represent F, Cl, —OCH₃, —OCH₂CH₃, —OCHF₂, —OCH₂CF₃, —OCF₃ or —OCF₂CF₃;
m, p and q independently represent 0, 1 or 2; and
n represents 0, 1, 2 or 3,
or a pharmaceutically-acceptable salt thereof,
provided that:
(A) when R¹ and R² both represent H and n represents 0, then X¹ does not represent a methyl substituent located at the 3- or 4-position of the 2-pyridyl ring,
(B) when R¹ represents H, R² represents methyl and n represents 0, then X¹ does not represent a methyl substituent located at the 4-position of the 2-pyridyl ring or a Br substituent located at the 5-position of the 2-pyridyl ring;
(C) when R¹ and R² both represent H and n represents 1, then X¹ and X² do not both represent methyl substituents located at the 4- and 6-positions of the 2-pyridyl ring,
which compounds and salts are referred to hereinafter as “the compounds of the invention”.

Pharmaceutically-acceptable salts include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Compounds of the invention may contain double bonds and may thus exist as E (entgegen) and Z (zusammen) geometric isomers about each individual double bond. All such isomers and mixtures thereof are included within the scope of the invention.

Compounds of the invention may also exhibit tautomerism. All tautomeric forms and mixtures thereof are included within the scope of the invention.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Unless otherwise specified, C_{1-q} alkyl (where q is the upper limit of the range), defined herein may be straight-chain or, when there is a sufficient number (i.e. a minimum of three) of carbon atoms, be branched-chain, and/or cyclic (so forming, in the case of alkyl, a C_{3-q} cycloalkyl group). Further, when there is a sufficient number (i.e. a mini mum of four) of carbon atoms, such groups may also be part cyclic. Further, unless otherwise specified, such alkyl groups may also be saturated or, when there is a sufficient number (i.e. a minimum of two) of carbon atoms and unless otherwise specified, be unsaturated (forming, for example, a C_{2-q} alkenyl or a C_{2-q} alkynyl group).

The term “halo”, when used herein, includes fluoro, chloro, bromo and iodo.

Heterocycloalkyl groups that may be mentioned include monocyclic or bicyclic heterocycloalkyl groups (which groups may further be bridged) in which at least one (e.g. one to four) of the atoms in the ring system is other than carbon (i.e. a heteroatom), and in which the total number of atoms in the ring system is between three and twelve (e.g. between five and ten). Further, such heterocycloalkyl groups may be saturated or unsaturated containing one or more double and/or triple bonds, forming for example a C_{2-q} heterocycloalkenyl (where q is the upper limit of the range) or a C_{3-q} heterocycloalkynyl group. C_{2-q} heterocycloalkyl groups that may be mentioned include 7-azabicyclo[2.2.1]heptanyl, 6-azabicyclo[3.1.1]hept-anyl, 6-azabicyclo[3.2.1]octanyl, 8-azabicyclo-[3.2.1]octanyl, aziridinyl, azetidinyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, 7-oxabicyclo[2.2.1]heptanyl, 6-oxabicyclo-[3.2.1]octanyl, oxetanyl, oxiranyl, piperazinyl, piperidinyl, pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuranyl, tetrahydropyridyl, thietanyl, thiiranyl, thiolanyl, thiomorpholinyl, trithianyl (including 1,3,5-trithianyl), tropanyl and the like. Substituents on heterocycloalkyl groups may, where appropriate, be located on any atom in the ring system including a heteroatom. Further, in the case where the other substituent is another cyclic compound, then the cyclic compound may be attached through a single atom on the heterocycloalkyl group, forming a so-called “spiro”-compound. The point of attachment of heterocycloalkyl groups may be via any atom in the ring system including (where appropriate) a heteroatom (such as a nitrogen atom), or an atom on any fused carbocyclic ring that may be present as part of the ring system. Heterocycloalkyl groups may also be in the N- or S-oxidised form.

Aryl groups that may be mentioned include C₆₋₁₄ (e.g. C₆₋₁₀) aryl groups. Such groups may be monocyclic, bicyclic or tricyclic and have between 6 and 14 ring carbon atoms, in which at least one ring is aromatic. C₆₋₁₄ aryl groups include phenyl, naphthyl and the like, such as 1,2,3,4-tetrahydronaphthyl, indanyl, indenyl and fluorenyl. The point of attachment of aryl groups may be via any atom of the ring system. However, when aryl groups are bicyclic or tricyclic, they are linked to the rest of the molecule via an atom of the aromatic ring.

Heteroaryl groups that may be mentioned include those which have between 5 and 14 (e.g. between 5 and 10) members. Such groups may be monocyclic, bicyclic or tricyclic, provided that at least one of the rings is aromatic and wherein at least one (e.g. one to four) of the atoms in the ring system is other than carbon (i.e. a heteroatom). Heteroaryl groups that may be mentioned include acridinyl, benzimidazolyl, benzodioxanyl, benzodioxepinyl, benzodioxolyl (including 1,3-benzodioxolyl), benzofuranyl, benzofurazanyl, benzothiazolyl, benzothiadiazolyl (including 2,3,1-benzothiadiazolyl), benzoxadiazolyl (including 2,1,3-benzoxadiazolyl), benzoxazinyl (including 3,4-dihydro-2H-1,4-benzoxazinyl), benzoxazolyl, benzimidazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2-a]pyridyl, indazolyl, indolinyl, indolyl, isobenzofuranyl, isochromanyl, isoindolinyl, isoindolyl, isoquinolinyl, isothiaziolyl, isothiochromanyl, isoxazolyl, naphthyridinyl (including 1,5-napthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. However, when heteroaryl groups are bicyclic or tricyclic, they are linked to the rest of the molecule via an atom of the aromatic ring. Heteroaryl groups may also be in the N- or S-oxidised form.

Heteroatoms that may be mentioned include phosphorus, silicon, boron, tellurium, selenium and, preferably, oxygen, nitrogen and sulphur.

For the avoidance of doubt, in cases in which the identity of two or more substituents in a compound of the invention may be the same, the actual identities of the respective substituents are not in any way interdependent. For example, in the situation in which the 2-pyridyl group of a compound of the invention is substituted by two or more substituents, those substituents may be the same or different. For example, when the 2-pyridyl ring of a compound of the invention is substituted by two substituents, and the substituents are both —C(O)R^{3b} in which R^{3b} is a Z group, the respective Z groups may be the same or different. Similarly, when the 2-pyridyl group is substituted by more than one substituent as defined herein, the identities of those individual substituents are not to be regarded as being interdependent. For example, when one substituent represents —C(O)R^{3b} and the other substituent represents —C(O)OR^{3c}, and R^{3b} and R^{3c} both represent C₁₋₆ alkyl substituted by —OR^{6d}, the identities of the two —OR^{6d} groups are not to be regarded as being interdependent.

Compounds of the invention that may be mentioned include those in which when R¹ and R² both represent H, n represents 0, then X¹ does not represent H.

Further compounds of the invention that may be mentioned include those in which:

when n represents 1, 2 or 3 and at least one of the X² substituents is located at the 3-position of the 2-pyridyl group, then X² represents:

### 1) G¹;

2) aryl or heteroaryl, both of which are optionally substituted by one or more substituents selected from A¹, —N₃, —NO₂ and —S(O)ₚR^{6e}; or
3) heterocycloalkyl, which is optionally substituted by one or more substituents selected from A², —N₃, —NO₂ and ═O,
in which the heteroaryl or heterocycloalkyl group does not contain a nitrogen atom and G¹ represents halo, —R^{3a}, —CN, —C(O)R^{3b}, —C(O)OR^{3c}, —C(O)N(R^{4a})R^{5a}, —N₃, —NO₂, —OR^{3h}, —OC(O)N(R^{4g})R^{5g}, —OS(O)₂R³ⁱ, —S(O)ₘR^{3j}, —OC(O)R³ⁿ, —OC(O)OR^{3p}, —S(O)₂N(R^{4h})R^{5h}, —S(O)₂OH, —P(O)(OR⁴ⁱ)(OR⁵ⁱ) or —C(O)N(R^{3q})S(O)₂R^{3r}.

Yet further compounds of the invention that may be mentioned include those in which:

when X¹ and/or (when n is 1, 2 or 3) X² is located at the 3-substituent, then X¹ represents halo and/or X² represents:

### 1) G¹;

2) aryl or heteroaryl, both of which are substituted by one or more substituents selected from A¹, —N₃, —NO₂ and —S(O)ₚR^{6e}; or
3) heterocycloalkyl, which is substituted by one or more substituents selected from A², —N₃, —NO₂ and ═O,
in which A¹ and A² independently represent —R^{6a}, —CN, —N(R^{6b})R^{6c} or —OR^{6d} and G¹ represents halo, —CN, —C(O)R^{3b}, —C(O)OR^{3c}, —C(O)N(R^{4a})R^{5a}, —N(R^{4b})R^{5b}, —N(R^{3d})C(O)R^{4c}, —N(R^{3e})C(O)N(R^{4d})R^{5d}, —N(R^{3f})C(O)OR^{4e}, —N₃, —NO₂, —N(R^{3g})S(O)₂N(R^{4f})R^{5f}, —OC(O)N(R^{4g})R^{5g}, —OS(O)₂R³ⁱ, —N(R^{3k})S(O)₂R^{3m}, —OC(O)R³ⁿ, —OC(O)OR^{3p}, —S(O)₂N(R^{4h})R^{5h}, —S(O)₂OH, —P(O)(OR⁴ⁱ)(OR⁵ⁱ) or —C(O)N(R^{3q})S(O)₂R^{3r}.

Yet further compounds of the invention that may be mentioned include those in which R^{4b} and R^{5b} are not linked together as defined herein.

Further compounds of the invention that may be mentioned include those in which:

the 2-pyridyl ring of the compound of formula I is not substituted (e.g. in the 5-position) by phenyl, 4H-[1,2,4]triazol-4-yl, pyridyl or indolizinyl;
when the 2-pyridyl group of the compound of formula I is substituted (for example in the 4-position) by a heteroaryl group, then such a heteroaryl group does not represent optionally substituted 4-pyrazolyl.

Preferred compounds of the invention include those in which:

R¹ and R² independently represent H, halo or C₁₋₃ alkyl (e.g. methyl) optionally substituted by one or more halo (e.g. fluoro) atoms (so forming, for example, a difluoromethyl or trifluoromethyl group);
R^{3k} and R^{3q} independently represent H;
R^{3m} and R^{3r} independently represent Z, in which Z represents aryl (e.g. phenyl), heteroaryl (e.g. pyridyl), which latter two groups are optionally substituted as defined herein, or C₁₋₆ (e.g. C₁₋₃) alkyl (e.g. methyl) optionally substituted by one or more fluoro atoms (so forming, for example, a trifluoromethyl group);
R^{3p} and R³ⁿ (when R³ⁿ represents optionally substituted alkyl) independently represent C₁₋₃ (e.g. C₁₋₂) alkyl optionally substituted by one or more fluoro atoms;
when Z represents an aryl or heteroaryl group, both of these are optionally substituted by one or more substituents selected from A⁴;
A¹, A², A³ and A⁴ independently represent halo (e.g. chloro or, particularly, fluoro), —R^{6a} or —OR^{6d};
when any of R^{6a} to R^{6e} or R^{7e} represent optionally substituted C₁₋₆ alkyl, then that alkyl group is an optionally substituted C₁₋₄ (e.g. C₁₋₂) alkyl group;
when R^{6b} and R^{6c} are linked together, they form a 5- to 6-membered ring, which ring optionally contains a further heteroatom (such as nitrogen or oxygen) and is optionally substituted by methyl, —CHF₂, —CF₃ or ═O (so forming, for example, a pyrrolidinyl, piperidinyl, morpholinyl or a piperazinyl (e.g. 4-methylpiperazinyl) ring);
B¹, B², B³ and B⁴ independently represent F or Cl;
m, p and q independently represent 2.

Further compounds of the invention that may be mentioned include those in which:

R¹ and R² independently represent H, F, Cl, CH₃, CHF₂ or CF₃;
n represents 2 or, more preferably, 1 or 0 (e.g. 0);
when any of the pairs R^{4a} and R^{5a}, R^{4b} and R^{5b}, R^{4d} and R^{5d}, R^{4f} and R^{5f}, R^{4g} and R^{5g}, R^{4h} and R^{5h}, or R^{6b} and R^{6c}, are linked together, they form a 5- to 6-membered ring, which ring optionally contains a further heteroatom (such as nitrogen or oxygen) and is optionally substituted by methyl, —CHF₂, —CF₃ or ═O (so forming, for example, a pyrrolidinyl, piperidinyl, morpholinyl or a piperazinyl (e.g. 4-methylpiperazinyl) ring);
R^{3a} represents C₁₋₆ alkyl optionally substituted by one or more substituents selected from F and —OR^{6d}.

Further preferred compounds of the invention include those in which:

X² (if present) and, more preferably, X¹ independently represent Br, ethyl, butyl, propyl, hydroxymethyl, iodo or, preferably, H, F, Cl, CH₃, CHF₂, CF₃, —OCH₃, —OCHF₂ and —OCF₃ (as appropriate);
when X² represents G¹, then G¹ represents —C(O)R^{3b}, —C(O)OR^{3c}, N(R^{3e})C(O)N(R^{4d})R^{5d}, —N(R^{3f})C(O)OR^{4e}, N₃, —NO₂, —N(R^{3g})S(O)₂N(R^{4f})R^{5f}, —OC(O)N(R^{4g})R^{5g}, —OS(O)₂R³ⁱ or, more preferably, Br, F, Cl, —R^{3a}, —CN, —C(O)N(R^{4a})R^{5a}, —N(R^{4b})R^{5b}, —N(R^{3d})C(O)R^{4c}, —OR^{3h}, —S(O)ₘR^{3j} or —S(O)₂N(R^{4h})R^{5h};
when X² represents aryl or heteroaryl, optional substituents are preferably selected from —NO₂ or, more preferably, A¹;
R^{3a} represents C₁₋₄ alkyl (such as butyl, propyl or, preferably, ethyl isopropyl, t-butyl, cyclopropyl, cyclobutyl, cyclopropylmethyl or, especially, methyl) optionally substituted by one or more substituents selected from —OR^{6d} or, preferably, fluoro (so forming, for example, a CHF₂ or CF₃ group);
R^{3b}, R^{3c}, R^{3h} and R^{4d} to R^{4h} independently represent H or C₁₋₄ alkyl optionally substituted by one or more substituents selected from halo and —OR^{6d};
R^{3d} to R^{3g} independently represent CH₃ or, more particularly, H;
R³ⁱ and R^{3j} independently represent C₁₋₄ (e.g. C₁₋₃) alkyl (such as methyl) optionally substituted by one or more B¹ substituents;
B¹ represents F (thus R³ⁱ and R^{3j} may represent a CH₃ or CF₃ group);
R^{4a} to R^{4c} independently represent H, Z or C₁₋₄ alkyl optionally substituted by one or more substituents selected from halo and —OR^{6d};
Z represents aryl or heteroaryl, both of which are optionally substituted by one or more substituents selected from —NO₂ or, preferably, A⁴;
A¹, A², A³ and A⁴ are independently selected from halo or —OR^{6d};
R^{5a}, R^{5b}, R^{5d} and R^{5f} to R⁵ⁱ independently represent H or C₁₋₄ alkyl optionally substituted by one or more substituents selected from halo and —OR^{6d}; or
the relevant pairs (i.e. R^{4a} and R^{5a}, R^{4b} and R^{5b}, R^{4d} and R^{5d}, R^{4f} and R^{5f}, R^{4g} and R^{5g} and R^{4h} and R^{5h}) are linked together as hereinbefore defined;
when any one of R^{3b}, R^{3c} to R^{3h}, R^{4a} to R^{4h}, R^{5a}, R^{5b}, R^{5d}, R^{5f} to R^{5h} represents alkyl, preferred optional substituents include —OCH₃ and, especially, F.

When the 2-pyridyl group of the compound of formula I is substituted by optionally substituted heterocycloalkyl, aryl or heteroaryl, then preferred values of such heterocycloalkyl, aryl or heteroaryl groups include optionally substituted indolyl (e.g. 4-indolyl), tetrazolyl, thienyl, triazolyl (e.g. 1,2,4-triazol-3-yl) or, more preferably, oxadiazolyl, oxazolyl, phenyl quinolinyl (e.g. 3-quinolinyl or 4-quinolinyl), pyrazolyl (e.g. 3-pyrazolyl), pyridyl (e.g. 2-pyridyl or 3-pyridyl), thiadiazolyl or thiazolyl.

Particularly preferred values of Z include optionally substituted indolyl (e.g. 4-indolyl), thienyl or, more preferably, oxadiazolyl, oxazolyl, phenyl, quinolinyl (e.g. 3-quinolinyl or 4-quinolinyl), pyrazolyl (e.g. 3-pyrazolyl), pyridyl (e.g. 2-pyridyl or 3-pyridyl), thiadiazolyl or thiazolyl.

Yet more preferred compounds of the invention include those in which:

when the 2-pyridyl group of the compound of formula I is substituted, then it is preferably substituted by one to three (e.g. one or two) substituents selected from aryl (e.g. phenyl) or, more preferably, G¹;
R^{3a} represents C₁₋₄ (such as C₁₋₃ (e.g. C₁₋₂)) alkyl (e.g. butyl, propyl or, preferably, methyl or ethyl) optionally substituted by one or more substituents selected from —OR^{6d} or, preferably, fluoro (so forming, for example, a —CF₃ group);
R^{3c} represents H or, more preferably, C₁₋₂ alkyl (e.g. methyl) optionally substituted by one or more fluoro atoms;
R^{3h} represents hydrogen or C₁₋₄ (such as C₁₋₃ (e.g. C₁₋₂)) alkyl (e.g. butyl, propyl or, preferably, methyl or ethyl) optionally substituted by one or more fluoro atoms (so forming, for example, a —CF₃ group);
R^{4b} and R^{5b} independently represent H or, more preferably, C₁₋₂ alkyl (e.g. methyl or ethyl);
G¹ represents —C(O)OH, —O—(CH₂)₃CH₃ (i.e. —O-n-butyl), —C(CH₂)₂CH₃ (i.e. —O-n-propyl), —NH₂, —N(H)C(O)-phenyl or, preferably, F, Cl, —CH₃, —CH₂CH₃, —CHF₂, —CF₃, —CH₂CF₃, —CN, —N(CH₃)₂, —N(CH₂CH₃)₂, —NO₂, —OH, —OCH₃, —OCH₂CH₃, —OCH₂CF₃, —OCHF₂, —OCF₃ or —OCF₂CF₃.

Preferred optional substituents on the 2-pyridyl ring of the compound of formula I include:

### —C(O)OR^{3c};

aryl optionally substituted by one or more groups selected from —NO₂ or, more particularly, A¹;

### —N(R^{4b})R^{5b};

### —N(R^{3d})C(O)R^{4c};

—CN; or, more preferably,

### —OR^{3h};

—NO₂; or, even more preferably,
halo (e.g. iodo or, preferably, bromo, fluoro or chloro);

### —R^{3a};

### —N(R^{3d})C(O)R^{4c};

R^{3a} represents C₁₋₄ alkyl (e.g. methyl), optionally substituted by one or more substituents selected from —OR^{6d} or, preferably, fluoro (so forming, for example, a CF₃ group);
R^{3c} represents C₁₋₂ alkyl substituted by one or more fluoro atoms or, preferably unsubstituted;
R^{3h} represents ethyl, butyl (e.g. n-butyl), propyl (e.g. n-propyl) or, more preferably, methyl;
R^{4c} represents aryl or, preferably, heteroaryl (such as a nitrogen-containing heteroaryl group (e.g. pyrazolyl));
R^{6d} represents C₁₋₂ alkyl or, more preferably, H.

Yet more preferred compounds of the invention include those in which:

R¹ and R² independently represent methyl optionally substituted by one or more fluoro atoms (so forming, for example, a difluoromethyl or trifluoromethyl group) or, more preferably, H, F or Cl;
X¹ represents Br, ethyl, butyl (e.g. n-butyl), propyl (e.g. n-propyl), hydroxymethyl (i.e. —CH₂OH), iodo, preferably H or, more preferably, F, Cl, CH₃ or CF₃;
X² represents aryl (e.g. phenyl; which group is substituted as hereinbefore defined or, preferably, unsubstituted) or, preferably, G¹;
n represents 0 or 1;
G¹ represents —NO₂, —OR^{3h}, —C(O)OR^{3c}, —N(R^{4b})R^{5b}, —CN, preferably R^{3a} or, more preferably, halo (e.g. Br) or —N(R^{3d})C(O)R^{4c};
R^{3c} represents methyl;
R^{3d} represents H;
R^{4c} represents aryl (e.g. unsubstituted phenyl) or, preferably, heteroaryl (e.g. pyrazolyl);
when the 2-pyridyl group of the compound of formula I is substituted, it is preferably substituted at the 5- and/or the 6-position.

Preferred compounds of the invention include those in which:

R¹ represents trifluoromethyl, preferably, chloro or, more preferably, H.
R² represents methyl, difluoromethyl, trifluoromethyl or, preferably, H or chloro;
when X¹ is other than H and/or at least one X² substituent is present, then it is preferred that one of these substituents is in the 3-, 4-, preferably 6- or, more preferably, 5-position of the 2-pyridyl ring.

Preferred substituents on the 2-pyridyl group of compounds of the invention include nitro, methoxy, ethyl, carboxymethyl, phenyl, butyl (e.g. n-butyl), ethoxy, butoxy (e.g. n-butoxy), propyl (e.g. n-propyl), hydroxymethyl, amino (e.g. —NH₂), cyano, propoxy (e.g. n-propoxy), benzamido and, more preferably, halo (e.g. iodo or, more particularly, chloro, bromo or fluoro), trifluoromethyl, methyl and pyrazole-3-carboxamido.

Preferred 2-pyridyl groups of compounds of the invention include 5-nitro-2-pyridyl, 5,6-dimethyl-2-pyridyl, 6-methoxy-2-pyridyl, 5-bromo-3-methyl-2-pyridyl, 5,6-dimethoxy-2-pyridyl, 3-methoxy-2-pyridyl, 5-ethyl-2-pyridyl, 5-carboxymethyl-2-pyridyl (i.e. methyl-6-nicotinate), 5-phenyl-2-pyridyl, 5-n-butyl-2-pyridyl, 5-ethoxy-2-pyridyl, 5-n-propoxy-2-pyridyl, 6-bromo-2-pyridyl, 5-bromo-2-pyridyl, 5-n-butoxy-2-pyridyl, 5-methoxy-2-pyridyl, 5-n-propyl-2-pyridyl, 5-ethyl-6-methyl-2-pyridyl, 5-hydroxymethyl-2-pyridyl, 3-chloro-5-trifluoromethyl-2-pyridyl, 6-trifluoromethyl-2-pyridyl, 6-methoxy-2-pyridyl, 4-methyl-2-pyridyl, 5-iodo-2-pyridyl, 5-methyl-2-pyridyl, 5-bromo-4-methyl-2-pyridyl, 6-methyl-2-pyridyl, 5-amino-2-pyridyl, 4,6-dimethyl-2-pyridyl, 5-cyano-2-pyridyl, 5-benzamido-2-pyridyl and more particularly, 5-chloro-2-pyridyl, 5-fluoro-2-pyridyl, 5-trifluoromethyl-2-pyridyl, 5-bromo-6-methyl-2-pyridyl and 5-(pyrazole-3-carboxamido)-2-pyridyl.

Particularly preferred compounds of the invention include those of the examples described hereinafter.

Compounds of the invention may be made in accordance with techniques that are well known to those skilled in the art, for example as described hereinafter.

According to a further aspect of the invention there is provided a process for the preparation of a compound of formula I, which process comprises:

(i) For compounds of formula I in which R² represents halo or C₁₋₆ alkyl, reaction of a corresponding compound of formula I in which R² represents hydrogen, with an appropriate base (or a mixture of bases), such as potassium bis(trimethylsilyl)amide, sodium bis(trimethylsilyl)amide, sodium hydride, potassium tert-butoxide or an organolithium base, such as n-BuLi, s-BuLi, t-BuLi, lithium diisopropylamide or lithium 2,2,6,6-tetramethylpiperidine (which organolithium base is optionally in the presence of an additive (for example, a lithium co-ordinating agent such as an ether (e.g. dimethoxyethane) or an amine (e.g. tetramethylethylenediamine (TMEDA), (−)sparteine or 1,3-dimethyl-3,4,5,6-tetrahydro-2(1H-pyrimidinone (DMPU) and the like)) followed by quenching with an appropriate electrophile such as:
(a) for compounds of formula I in which R² represents an optionally substituted C₁₋₆ alkyl group, a compound of formula II,

R^{c}L^{1a} II
wherein R^{c} represents C₁₋₆ alkyl (which alkyl group is optionally substituted by one or more halo atoms), and L^{1a} represents a suitable leaving group such as halo (e.g. iodo or bromo) or a sulfonate group (such as —OSO₂CF₃, OSO₂CH₃ and —OSO₂-aryl (e.g. —O-tosyl)) or, for compounds of formula I in which R² represents CF₃, a trifluoromethylating reagent, such as 5-(trifluoromethyl)-dibenzothiophenium tetrafluoroborate; or
(b) for compounds of formula I in which R² represents halo, an electrophile that provides a source of these atoms. For example, for bromine atoms, reagents include N-bromosuccinimide, bromine and 1,2-dibromotetrachloroethane, for chlorine atoms reagents include N-chlorosuccinimide, chlorine, iodine monochloride and hexachloroethane, for iodine atoms, appropriate reagents include iodine, diiodoethane and diiodotetrachloroethane and for fluorine atoms reagents include xenon difluoride, SELECTFLUOR® ([1-(chloromethyl)-4-fluoro-1,4-diazonia-bicyclo[2.2.2]octane bis(tetrafluoroborate)]), CF₃OF, perchloryl fluoride, F₂ and acetylhypofluoride.

The skilled person will appreciate that the corresponding compounds of formula I in which R² represents hydrogen (on which the above reaction is performed) may need to be protected at the nitrogen atom of the pyrazole ring system, preferably with a protective group that is also a directing metallation group (such as a benzenesulfonyl group or a SEM (i.e. a —CH₂OC₂H₄Si(CH₃)₃) group). The reaction may be performed in the presence of a suitable solvent, such as a polar aprotic solvent (e.g. tetrahydrofuran or diethyl ether), at sub-ambient temperatures (e.g. 0° C. to −78° C.) under an inert atmosphere followed (as appropriate) by deprotection of the N-protective group under standard conditions (e.g. when a benzenesulfonyl group is employed, by hydrolysis or, when a SEM group is employed by reaction in the presence of HCl in ethanol).

(ii) For compounds of formula I in which R² represents CF₃, reaction of a corresponding compound of formula I in which R² represents bromo or, preferably, iodo with CuCF₃ (or a source of CuCF₃) in, for example, the presence of HMPA and DMF. The skilled person will appreciate that the reagent CuCF₃ may not be isolated as such, and may be prepared in accordance with the procedures described in Burton D. G.; Wiemers D. M., J. Am. Chem. Soc., 1985, 107, 5014-5015 and Mawson S. D.; Weavers R. T.; Tetrahedron Letters., 1993, Vol. 34, No. 19, 3139-3140 (for example, by the reaction of zinc and e.g. CF₂Br₂ in DMF so forming ZnCF₃ (or a source thereof) followed by treatment with CuBr in HMPA).
(iii) Reaction of a compound of formula III,

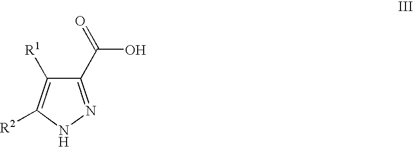

or a N-protected and/or O-protected (e.g. ester) derivative thereof, wherein R¹ and R² are as hereinbefore defined, with a compound of formula IV,

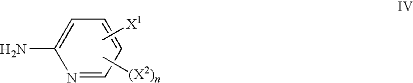

wherein X¹, X² and n are as hereinbefore defined under coupling conditions, for example at around room temperature or above (e.g. up to 40-180° C.), optionally in the presence of a suitable base (e.g. sodium hydride, sodium bicarbonate, potassium carbonate, pyrrolidinopyridine, pyridine, triethylamine, tributylamine, trimethylamine, dimethylaminopyridine, diisopropylamine, diisopropylethylamine, 1,8-diazabicyclo[5.4.0]undec-7-ene, sodium hydroxide, N-ethyldiisopropylamine, N-(methylpolystyrene)-4-(methylamino)pyridine, butyllithium (e.g. n-, s- or t-butyllithium) or mixtures thereof), an appropriate solvent (e.g. tetrahydrofuran, pyridine, toluene, dichloromethane, chloroform, acetonitrile, dimethylformamide, dimethylsulfoxide, water or triethylamine) and a suitable coupling agent (e.g. 1,1′-carbonyldiimidazole, N,N′-dicyclohexylcarbodiimide, 1-(3-dimethylamino-propyl)-3-ethylcarbodiimide (or hydrochloride thereof), N,N′-disuccinimidyl carbonate, benzotriazol-1-yloxytris(dimethylamino) phosphonium hexafluoro-phosphate, 2-(1H-benzotriazol-1-yl)-1,1,3,3-tetramethyluronium hexafluorophos-phate, benzotriazol-1-yloxytrispyrrolidinophosphonium hexafluorophosphate, bromo-tris-pyrrolidinophosphonium hexafluorophosphate, 2-(1H-benzotriazol-1-yl)-1,1,3,3-tetramethyluronium tetrafluorocarbonate, 1-cyclohexyl-carbodiimide-3-propyloxymethyl polystyrene, O-(7-azabenzotriazol-1-yl)-N,N,N′,N′-tetramethyluronium hexafluorophosphate or O-benzotriazol-1-yl-N,N,N′,N′-tetramethyluronium tetrafluoroborate). Alternatively, compounds of formula III may first be activated by treatment with a suitable reagent (e.g. oxalyl chloride, thionyl chloride, etc) optionally in the presence of an appropriate solvent (e.g. dichloromethane, dimethylformamide, THF, toluene or benzene) and a suitable catalyst (e.g. DMF), resulting in the formation of the respective acyl chloride. This activated intermediate may then be reacted with a compound of formula IV under standard conditions, such as those described above. The skilled person will appreciate that when compounds of formula I are liquid in nature, they may serve as both solvent and reactant in this reaction. Alternative methods of performing this step include reaction of an O-protected derivative (e.g. an ethyl ester) of a compound of formula III with a compound of formula IV, which latter compound may first be treated with an appropriate reagent (e.g. trimethylaluminum), for example in an inert atmosphere and in the presence of a suitable solvent (e.g. dichloromethane).
(iv) Reaction of a compound of formula V,

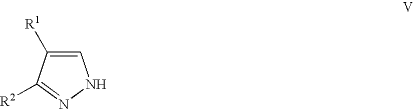

wherein R¹ and R² are as hereinbefore defined, with a suitable base, such as one described in process step (i) above, followed by reaction with a compound of formula VI,

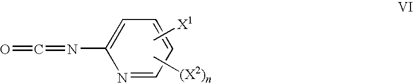

wherein X¹, X² and n are as hereinbefore defined, followed by quenching with a suitable proton source (e.g. water or aqueous, saturated NH₄Cl solution). This reaction may be performed under similar conditions to those described above in respect of process step (i). The skilled person will therefore appreciate that the pyrazole nitrogen may need to be protected.
(v) For compounds of formula I in which R² represents hydrogen and R¹ is as hereinbefore defined, removal of the group J from a compound of formula VII,

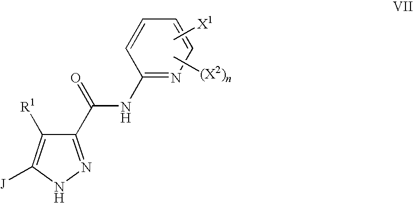

wherein J represents —Si(R^{t})₃ or —Sn(R^{z})₃ (in which each R^{t} independently represents a C₁₋₆ alkyl (e.g. a methyl or isopropyl) group or an aryl (e.g. phenyl) group and each R^{z} independently represents C₁₋₆ alkyl (e.g. methyl or butyl)), and R¹, X¹, X² and n are as hereinbefore defined. When J represents —Si(R^{t})₃, the reaction may be performed in the presence of an appropriate reagent for the removal of the silyl group, such as a source of halide anions (e.g. tetrabutylammonium fluoride, tetramethylammonium fluoride, hydrogen fluoride or potassium fluoride), for example, in the presence of a suitable solvent (e.g. tetrahydrofuran) at room temperature. When J represents —Sn(R^{z})₃, the reaction may be a standard hydrolysis, for example reaction with water or an aqueous acid (e.g. hydrochloric acid) in the presence of an appropriate solvent (e.g. dioxane, tetrahydrofuran, methanol or ethanol (or mixtures thereof)).
(vi) Reaction of a compound of formula VIII,

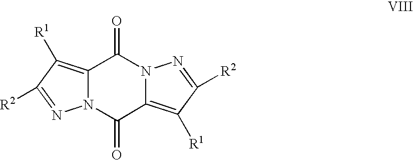

wherein R¹ and R² are as hereinbefore defined, with a compound of formula IV as hereinbefore defined, for example under coupling conditions such as those described hereinbefore in respect of process step (iii) above. Preferred conditions include reaction in the presence of base, solvent but no coupling reagent. In this case, the compound of formula IV may also be employed in excess.
(vii) For compounds of formula I in which one of R¹ or R² represents halo or C₁₋₆ alkyl optionally substituted as hereinbefore defined, and the other represents H, reaction of a corresponding compound of formula I in which one of R¹ or R² represents bromo or iodo and the other represents H (as appropriate) with a suitable organolithium base (e.g. t-BuLi, s-BuLi or n-BuLi) optionally in the presence of an additive (such as one hereinbefore described in respect of process step (i)), followed by quenching with a compound of formula II, as hereinbefore defined, or a source of halogen atoms, such as one described in respect of process (i) above. This reaction may be performed in the presence of a suitable solvent, such as one hereinbefore described in respect of process step (i) at low temperatures (e.g. −78 to −120° C.) under an inert atmosphere.
(viii) Reaction of a compound of formula VIIIA

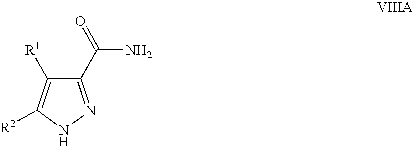

or a N-protected (e.g. at the pyrazole nitrogen) derivative thereof, wherein R¹ and R² are as hereinbefore defined, with a compound of formula VIIIB,

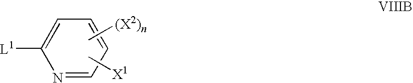

wherein L¹ represents a suitable leaving group, such as halo (e.g. chloro, bromo and iodo), —OSO₂CF₃, —B(OH)₂, —Sn(R^{z})₃ (wherein R^{z} is as hereinbefore defined), —Pb(OC(O)CH₃)₃, —Bi(W)₂, —Bi(W)₂(OC(O)CH₃)₂, —Bi(W)₂(OC(O)CF₃)₂ or —I(W)(BF₄), and W represents an aryl or heteroaryl group, both of which are optionally substituted by one or more groups selected from X² as hereinbefore defined (e.g. W represents the phenyl ring of the compound of formula I as hereinbefore defined), and X¹, X² and n are as hereinbefore defined, for example in the presence of a catalyst containing, preferably, Pd or Cu, and a base, such as potassium or sodium hydroxide, potassium carbonate, potassium tert-butoxide and lithium N,N-diisopropylamide. Catalysts that may be mentioned include Pd₂(dba)₃ (tris(dibenzylideneacetone)dipalladium(0)), bases that may be mentioned include cesium carbonate, ligands that may be mentioned include 2,2′-bis(diphenylphosphino)-1,1′-binaphthyl and solvents that may be employed include toluene. Such reactions may be performed at elevated temperature (e.g. at about 90° C.) under an inert (e.g. argon) atmosphere.

Compounds of formula III (or derivatives thereof) in which R² represents H or C₁₋₆ alkyl optionally substituted by one or more halo atoms may be prepared by reaction of a compound of formula IX,

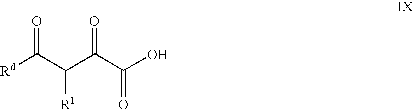

or an enol ether equivalent (e.g. a methyl enol ether or a silyl (e.g. trimethylsilyl) enol ether), or an O-protected (e.g. at the carboxylic acid) derivative thereof, wherein R^{d} represents H or C₁₋₆ alkyl optionally substituted by one or more halo atoms and R¹ is as hereinbefore defined, with hydrazine (or a hydrate or derivative (e.g. benzylhydrazine) thereof), for example in the presence of an alcoholic solvent (e.g. ethanol) at elevated temperature (e.g. at reflux).

Compounds of formula III in which either one of R¹ or R² represents halo and the other represents H or optionally substituted C₁₋₆ alkyl or both R¹ and R² represent halo, may be prepared by reaction of a corresponding compound of formula III in which R¹ and R² both represent H or one of R¹ or R² represents H and the other represents optionally substituted C₁₋₆ alkyl, with an electrophile that provides a source of halogen atoms, such as one described hereinbefore in respect of process step (i)(b), under reaction conditions known to those skilled in the art such as in the presence of a suitable solvent (e.g. water). Thus, relevant 4-halo, 5-halo or 4,5-dihalo substituted pyrazoles may be prepared in such a manner.

Compounds of formula III in which one of R¹ or R² represents fluoro and the other represents H may be prepared from 4-nitropyrazole-3-carboxylic acid or 5-nitropyrazole-3-carboxylic acid (as appropriate) employing an appropriate reagent for the conversion of the nitro group to a fluoro group (such as sodium fluoride, potassium fluoride, tetramethylammonium fluoride or tetrabutylammonium fluoride) under conditions known to those skilled in the art.

Compounds of formula III in which one of R¹ or R² represents halo and the other represents H, may be prepared by reaction of a compound of formula III in which one of R¹ or R² represents amino and the other represents H (as appropriate) followed by conversion of the amino group to a diazonium salt (employing reagents and conditions known to those skilled in the art, e.g. NaNO₂ and HCl at 5° C.) and then the addition of an appropriate nucleophile for the conversion to a halo group. Suitable nucleophiles include potassium, sodium or copper halides. Alternatively, for the introduction of the fluoro group, the appropriate diazonium salt may be treated with a compound that provides a source of fluoroborate (e.g. tetrafluoroborate) salts, for example by introducing a cold aqueous solution of NaBF₄, HBF₄ or NH₄BF₄, so forming the appropriate diazonium fluoroborate (e.g. diazonium tetrafluorborate), which may then be heated.

Compounds of formula III in which R¹ represents halo (e.g. F or Cl) or C₁₋₆ alkyl optionally substituted by one or more halo atoms may be prepared from corresponding compounds of formula III in which R¹ represents H, for example in accordance with a procedure described in R. Storer et al., Nucleosides & Nucleotides 18, 203 (1999). The appropriate reagents that may be employed for the introduction of the halo or optionally substituted C₁₋₆ alkyl group are described hereinbefore in respect of preparation of compounds of formula I (process step (i) above).

Compounds of formula III in which R¹ and R² independently represent perfluoro-C₁₋₆ alkyl or, preferably, H or halo may alternatively be prepared by oxidation of a compound of formula X,

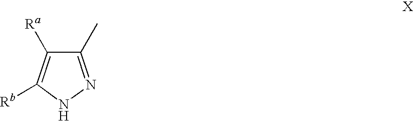

wherein R^{a} and R^{b} independently represent perfluoro-C₁₋₆ alkyl or, preferably, H or halo, under oxidation conditions known to those skilled in the art, for example mild or strong (e.g. employing an aqueous solution of potassium permanganate and heating at reflux) oxidation conditions as appropriate.

Compounds of formula III in which R² is as hereinbefore defined (e.g. halo) may be prepared by reaction of a compound of formula XI,

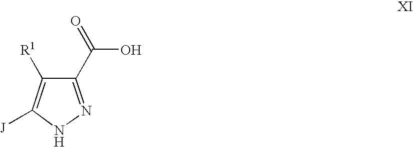

or a N-protected and/or O-protected (e.g. ester) derivative thereof, wherein J and R¹ are as hereinbefore defined. For compounds of formula III in which R² represents halo, reaction may be with a suitable halogenating reagent such as cesium fluoroxysulfate (in the case of a fluorinating reagent) or one described hereinbefore in respect of process step (i)(b), optionally in the presence of a suitable solvent (e.g. hexane, diethyl ether, tetrahydrofuran or 1,4-dioxane or mixtures thereof) under conditions known to those skilled in the art. For compounds of formula III in which R² represents H, reaction may be with reagents and under conditions such as those hereinbefore described in respect of preparation of compounds of formula I (process step (v)).

Compounds of formula III in which R¹ and R² are as hereinbefore defined may be prepared by oxidation of a compound of formula XIA,

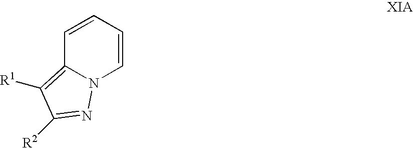

wherein R¹ and R² are as hereinbefore defined, under oxidation conditions known to those skilled in the art, such as those described hereinbefore in respect of preparation of compounds of formula III (i.e. from a compound of formula X) above.

Compounds of formula III (or protected derivatives thereof) in which R² represents H and R¹ is as hereinbefore defined (and preferably represents H or C₁₋₆ alkyl optionally substituted as hereinbefore defined) and may be prepared by reaction of a compound of formula XIB,

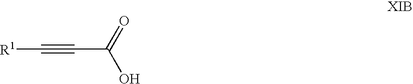

or a protected derivative (e.g. an ester, such as a C₁₋₆ (e.g. ethyl) ester) thereof, wherein R¹ is as hereinbefore defined (and preferably represents H or C₁₋₆ alkyl optionally substituted as hereinbefore defined), with diazomethane, or a protected derivative thereof (e.g. trimethylsilyldiazomethane), for example under conditions known to those skilled in the art (such as in the presence of a suitable solvent (e.g. diethyl ether) and/or at low temperatures (e.g. 0° C. to room temperature)).

Compounds of formula III or X may be prepared by reaction of a corresponding compound of formula V (e.g. for preparation of compounds of formula X, a compound of formula V in which R¹ and R² represent R^{a} and R^{b}, respectively) with a suitable base, such as one described in respect of preparation of compounds of formula I, process step (i) (and, in particular, organolithiums) followed by reaction with an appropriate electrophile. For example, in the case of compounds of formula III, for the introduction of a carboxylic acid group (or a protected derivative thereof), the electrophile may be a source of CO₂ (e.g. CO₂ gas), which addition is followed by the addition of a suitable proton source (e.g. HCl), or a compound of formula XV as defined hereinafter (e.g. methyl or ethyl chloroformate) or, in the case of compounds of formula X, a compound of formula XVI as defined hereinafter (e.g. methyl iodide), or the like.

Compounds of formula IV in which n is other then 0 and X² represents G¹ in which G¹ represents —S(O)₂N(R^{4h})R^{5h} and X¹ is as hereinbefore defined, may be prepared by reaction of a compound of formula XIC,

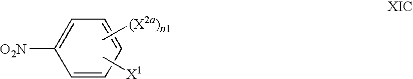

wherein n1 represents other then 0 and X^{2a} represents G¹ in which G¹ represents —S(O)₂N(R^{4h})R^{5h} and X¹ is as hereinbefore defined with a compound of formula XID,

H₂N(R^{4h})R^{5h} XID

wherein R^{4h} and R^{5h} are as hereinbefore defined, for example under conditions known to those skilled in the art (such as in the presence of a suitable base (e.g. triethylamine) and a suitable solvent (e.g. dichloromethane)), followed by hydrogenation of the isolated nitro intermediate, for example under conditions known to those skilled in the art (such as in the presence of a suitable catalyst (e.g. Pd on carbon (10%)) and a suitable solvent (e.g. methanol)).

Compounds of formula V may be prepared from a compound of formula XIE,

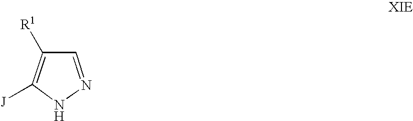

or a N-protected derivative thereof wherein J and R¹ are as hereinbefore defined, using reagents and procedures known to those skilled in the art, for example such as those hereinbefore described in respect of preparation of compounds of formula I (process route (v)), or in respect of preparation of compounds of formula III (the process involving reaction with a compound of formula XI).

Compounds of formula VII may be prepared by reaction of a compound of formula IV as hereinbefore defined with either.

(I) a compound of formula XII,

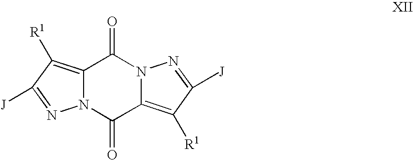

wherein R¹ and J are as hereinbefore defined; or
(II) a compound of formula XI as hereinbefore defined (or a N-protected and/or O-protected (e.g. ester) derivative thereof), for example under coupling conditions similar to those described hereinbefore in respect of preparation of compounds of formula I (process step (iii) or (vi) above).

Compounds of formulae VIII and XII may be prepared from compounds of formula III, and compounds of formula XI, respectively, under dimerising conditions, for example in the presence of thionyl chloride or oxalyl chloride (optionally in the presence of a suitable solvent and catalyst, such as one hereinbefore defined in respect of process step (iii)). Other dimerising reagents include carbodiimides, such as 1,3-dicyclohexylcarbodiimide or 1-(3-dimethylaminopropyl)-3-ethylcarbodiimide (EDCI, or hydrochloride thereof) optionally in the presence of a suitable base (e.g. 4-dimethylaminopyridine).

Compounds of formula X in which R^{b} represents halo may be prepared from a corresponding compound of formula X (or a protected derivative thereof) in which R^{b} represent H, for example under conditions and employing reagents such as those described hereinbefore in respect of preparation of compounds of formula I (process step (i) above).

Alternatively, compounds of formula X may be prepared by N-dealkylation of a compound of formula XIIA,

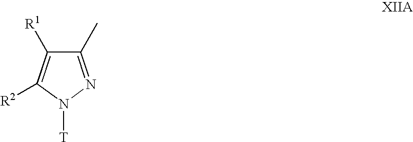

wherein T represents optionally substituted C₁₋₆ alkyl (e.g. methyl) and R¹ and R² are as hereinbefore defined, under dealkylation conditions known to those skilled in the art, for example by reaction with a suitable reagent (e.g. pyridine hydrochloride) at high temperatures (e.g. 150° C. to 220° C.)). Such a reaction may be carried out in the presence of a suitable solvent, but preferably no further solvent is present.

Alternatively, compounds of formula X may be prepared from a compound of formula XIIB,

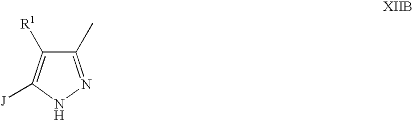

or a N-protected derivative thereof, wherein J and R¹ are as hereinbefore defined, using reagents and procedures known to those skilled in the art, for example such as those hereinbefore described in respect of preparation of compounds of formula I (process route (v)), or in respect of preparation of compounds of formula III (the process involving reaction with a compound of formula XI).

Compounds of formula XI (or a N-protected and/or O-protected (e.g. ester) derivative thereof) in which R¹ is as hereinbefore defined and preferably represents H or C₁₋₆ alkyl optionally substituted with one or more halo atoms may be prepared by reaction of a compound of formula XIII,

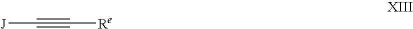

wherein R^{e} represents R¹ as hereinbefore defined and preferably, H or C₁₋₆ alkyl optionally substituted with one or more halo atoms and J is as hereinbefore defined, with a compound of formula XIV,

N₂—C(H)—C(O)OH XIV

or a O-protected (e.g. ester) derivative thereof, for example at elevated temperature (e.g. at between 80 and 120° C.) for between 1 and 3 days, optionally in the presence of an inert gas and preferably without the presence of solvent.

Compounds of formula XI (or a N-protected and/or C-protected (e.g. ester) derivative thereof) in which R¹ and J are as hereinbefore defined may be prepared by oxidation of a compound of formula XIIB as hereinbefore defined, under oxidation conditions known to those skilled in the art, for example such as those hereinbefore described in respect of preparation of compounds of formula III (the process involving reaction with a compound of formula X).

Alternatively, compounds of formula XI and XIIB (or, where applicable, a N-protected and/or O-protected (e.g. ester) derivative thereof) in which R¹ and J are as hereinbefore defined may be prepared by reaction of a compound of formula XIE, as hereinbefore defined, with an appropriate base (or a mixtures of bases), such as those described in respect of preparation of compounds of formula I process (i) above), followed by quenching with an appropriate electrophile such as:
(a) for compounds of formula XI, a source of CO₂ (e.g. CO₂ gas; which addition is followed by the addition of a suitable proton source (e.g. HCl), or a compound of formula XV,

R^{f}C(O)OL^{1c} XV
wherein R^{f} represents C₁₋₆ alkyl and L^{1c} represents a suitable leaving group such as halo (e.g. iodo, bromo or chloro); or
(b) for compounds of formula XIIB, a compound of formula XVI,

CH₃L^{1d} XVI
or the like (i.e. another suitable mediating reagent), wherein L^{1d} represents a suitable leaving group such as halo (e.g. iodo or bromo) or a sulfonate group (such as —OSO₂CF₃, OSO₂CH₃ and —OSO₂-aryl (e.g. —O-tosyl)).

Compounds of formula XIA may be prepared by reaction of 1-aminopyridinium iodide with a compound of formula XVII,

(R¹)(Cl)C═C(Cl)(R²) XVII

wherein R¹ and R² are as hereinbefore defined and the geometry of the double bond may be cis or trans, for example under conditions known to those skilled in the art (such as in the presence of a suitable base (e.g. potassium carbonate) and a suitable solvent (e.g. THF)). The skilled person will appreciate that the geometry around the double bond may affect the regioselectivity of the reaction

Compounds of formula XIE may be prepared by reaction of a compound of formula XVIII,

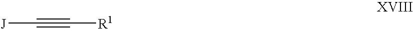

wherein R¹ and J are as defined hereinbefore, with diazomethane under conditions known to those skilled in the art, for example, in accordance with procedures described in T. Hanamoto et al., Chem. Commun, 2041 (2005), e.g. in the presence of a suitable solvent (e.g. hexane, diethyl ether, tetrahydrofuran or 1,4-dioxane or mixtures thereof) and optionally in the presence of an inert gas.

Compounds of formulae II, IV, V, VI, VIIIA, VIIIB, IX, XIB, XIC, XID, XIIA, XIII, XIV, XV, XVI, XVII and XVIII are either commercially available, are known in the literature, or may be obtained either by analogy with the processes described herein, or by conventional synthetic procedures, in accordance with standard techniques, from available starring materials using appropriate reagents and reaction conditions. In this respect, the skilled person may refer to inter alia “Comprehensive Organic Synthesis” by B. M. Trost and I. Fleming, Pergamon Press, 1991.

The substituents X¹ and X² (if present) as hereinbefore defined may be modified one or more times, after or during the processes described above for preparation of compounds of formula I by way of methods that are well known to those skilled in the art. Examples of such methods include substitutions, reductions, oxidations, alkylations, acylations, hydrolyses, esterifications and etherifications. The precursor groups can be changed to a different such group, or to the groups defined in formula I, at any time during the reaction sequence. In the case where the substituent X¹ and/or X² represents a halo group, such groups may be inter-converted one or more times, after or during the processes described above for the preparation of compounds of formula I. Appropriate reagents include NiCl₂ (for the conversion to a chloro group). In this respect, the skilled person may also refer to “Comprehensive Organic Functional Group Transformations” by A. R. Katritzky, O. Meth-Cohn and C. W. Rees, Pergamon Press, 1995.

Other transformations that may be mentioned include the conversion of a halo group (preferably iodo or bromo) to a cyano or 1-alkynyl group (e.g. by reaction with a compound which is a source of cyano anions (e.g. sodium, potassium, copper (I) or zinc cyanide) or with a 1-alkyne, as appropriate). The latter reaction may be performed in the presence of a suitable coupling catalyst (e.g. a palladium and/or a copper based catalyst) and a suitable base (e.g. a tri-(C₁₋₆ allyl)amine such as triethylamine, tributylamine or ethyldiisopropylamine). Further, amino groups and hydroxy groups may be introduced in accordance with standard conditions using reagents known to those skilled in the art.

Compounds of the invention may be isolated from their reaction mixtures using conventional techniques.

It will be appreciated by those skilled in the art that, in the processes described above and hereinafter, the functional groups of intermediate compounds may need to be protected by protecting groups. For example the pyrazole nitrogen or (when there is an —N(R^{4b})R^{5b} substituent on the 2-pyridyl ring of a compound of the invention) the nitrogen of the —N(R^{4b})R^{5b} group may need to be protected. Suitable nitrogen-protecting groups include those which form:

(i) carbamate groups (i.e. alkoxy- or aryloxy-carbonyl groups);
(ii) amide groups (e.g. acetyl groups);
(iii) N-alkyl groups (benzyl or SEM groups);
(iv) N-sulfonyl groups (e.g. N-arylsulfonyl groups);
(v) N-phosphinyl and N-phosphoryl groups (e.g. diarylphosphinyl and diarylphosphoryl groups); or
(vi) N-silyl group (e.g. a N-trimethylsilyl group).
Further, the skilled person will appreciate that, in the case where there are two functional groups protected (e.g. in the case where the carboxylic acid group of the compound of formula III is an ester and the pyrazole nitrogen is protected with a benzenesulfonyl group, then both groups may be deprotected in one step (e.g. a hydrolysis step known to those skilled in the art).

Further protecting groups for the pyrazole nitrogen include a methyl group, which methyl group may be deprotected under standard conditions, such as employing a pyridine hydrochloride salt at elevated temperature, for example using microwave irradiation in a sealed vessel at 200° C.

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques.

The type of chemistry involved will dictate the need, and type, of protecting groups as well as the sequence for accomplishing the synthesis.

The use of protecting groups is fully described in “Protective Groups in Organic Chemistry”, edited by J W F McOmie, Plenum Press (1973), and “Protective Groups in Organic Synthesis”, 3rd edition, T. W. Greene & P. G. M. Wutz, Wiley-Interscience (1999).

### Medical and Pharmaceutical Uses

Compounds of the invention are useful because they possess pharmacological activity. Such compounds are therefore indicated as pharmaceuticals. According to a further aspect of the invention there is provided a compound of formula I, as hereinbefore defined but without the provisos, or a pharmaceutically-acceptable salt thereof, for use as a pharmaceutical and/or in isolated (i.e. ex vivo) form.

Although compounds of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the “active” compounds to which they are metabolised), may therefore be described as “prodrugs” of compounds of the invention. All prodrugs of compounds of the invention are included within the scope of the invention.

By “prodrug of a compound of the invention”, we include compounds that form a compound of the invention, in an experimentally-detectable amount, within a predetermined time (e.g. about 1 hour), following oral or parenteral administration.

Compounds of the invention are useful because, in particular, they may inhibit the activity of lipoxygenases (and particularly 15-lipoxygenase), i.e. they prevent the action of 15-lipoxygenase or a complex of which the 15-lipoxygenase enzyme forms a part and/or may elicit a 15-lipoxygenase modulating effect, for example as may be demonstrated in the test described below. Compounds of the invention may thus be useful in the treatment of those conditions in which inhibition of a lipoxygenase, and particularly 15-lipoxygenase, is required.

Compounds of the invention are thus expected to be useful in the treatment of inflammation.

The term “inflammation” will be understood by those skilled in the art to include any condition characterised by a localised or a systemic protective response, which may be elicited by physical trauma, infection, chronic diseases, such as those mentioned hereinbefore, and/or chemical and/or physiological reactions to external stimuli (e.g. as part of an allergic response). Any such response, which may serve to destroy, dilute or sequester both the injurious agent and the injured tissue, may be manifest by, for example, heat, swelling, pain, redness, dilation of blood vessels and/or increased blood flow, invasion of the affected area by white blood cells, loss of function and/or any other symptoms known to be associated with inflammatory conditions.

The term “inflammation” will thus also be understood to include any inflammatory disease, disorder or condition per se, any condition that has an inflammatory component associated with it, and/or any condition characterised by inflammation as a symptom, including inter alia acute, chronic, ulcerative, specific, allergic and necrotic inflammation, and other forms of inflammation known to those skilled in the art. The term thus also includes, for the purposes of this invention, inflammatory pain and/or fever.

Accordingly, compounds of the invention may be useful in the treatment of asthma, chronic obstructive pulmonary disease (COPD), pulmonary fibrosis, allergic disorders, rhinitis, inflammatory bowel disease, ulcers, inflammatory pain, fever, atherosclerosis, coronary artery disease, vasculitis, pancreatitis, arthritis, osteoarthritis, rheumatoid arthritis, conjunctivitis, iritis, scleritis, uveitis, wound healing, dermatitis, eczema, psoriasis, stroke, diabetes, autoimmune diseases, Alzheimer's disease, multiple sclerosis, sarcoidosis, Hodgkin's disease and other malignancies, and any other disease with an inflammatory component.

Compounds of the invention may also have effects that are not linked to inflammatory mechanisms, such as in the reduction of bone loss in a subject. Conditions that may be mentioned in this regard include osteoporosis, osteoarthritis, Paget's disease and/or periodontal diseases. Compounds of formula I and pharmaceutically acceptable salts thereof may thus also be useful in increasing bone mineral density, as well as the reduction in incidence and/or healing of fractures, in subjects.

Compounds of the invention are indicated both in the therapeutic and/or prophylactic treatment of the above-mentioned conditions.

According to a further aspect of the present invention, there is provided a method of treatment of a disease which is associated with, and/or which can be modulated by inhibition of, a lipoxygenase (such as 15-lipoxygenase), and/or a method of treatment of a disease in which inhibition of the activity of a lipoxygenase, and particularly 15-lipoxygenase, is desired and/or required (e.g. inflammation), which method comprises administration of a therapeutically effective amount of a compound of formula I as hereinbefore defined but without the provisos, or a pharmaceutically-acceptable salt thereof, to a patient suffering from, or susceptible to, such a condition.

“Patients” include mammalian (including human) patients.

The term “effective amount” refers to an amount of a compound, which confers a therapeutic effect on the treated patient. The effect may be objective (i.e. measurable by some test or marker) or subjective (i.e. the subject gives an indication of or feels an effect).

Compounds of the invention will normally be administered orally, intravenously, subcutaneously, buccally, rectally, dermally, nasally, tracheally, bronchially, sublingually, by any other parenteral route or via inhalation, in a pharmaceutically acceptable dosage form.

Compounds of the invention may be administered alone, but are preferably administered by way of known pharmaceutical formulations, including tablets, capsules or elixirs for oral administration, suppositories for rectal administration, sterile solutions or suspensions for parenteral or intramuscular administration, and the like.

Such formulations may be prepared in accordance with standard and/or accepted pharmaceutical practice.

According to a further aspect of the invention there is thus provided a pharmaceutical formulation including a compound of formula I, as hereinbefore defined but without the provisos, or a pharmaceutically-acceptable salt thereof, in admixture with a pharmaceutically acceptable adjuvant, diluent or carrier.

The invention further provides a process for the preparation of a pharmaceutical formulation, as hereinbefore defined, which process comprises bringing into association a compound of formula I, as hereinbefore defined but without the provisos, or a pharmaceutically acceptable salt thereof with a pharmaceutically-acceptable adjuvant, diluent or carrier.

Compounds of the invention may also be combined with other therapeutic agents that are useful in the treatment of inflammation as defined herein (e.g. NSAIDs, coxibs, corticosteroids, analgesics, inhibitors of 5-lipoxygenase, inhibitors of FLAP (5-lipoxygenase activating protein), and leukotriene receptor antagonists (LTRas), and/or other therapeutic agents that are useful in the treatment of inflammation).

According to a further aspect of the invention, there is provided a combination product comprising:
(A) a compound of formula I, as hereinbefore defined but without the provisos, or a pharmaceutically-acceptable salt thereof; and
(B) another therapeutic agent that is useful in the treatment of inflammation,
   wherein each of components (A) and (B) is formulated in admixture with a pharmaceutically-acceptable adjuvant, diluent or carrier.

Such combination products provide for the administration of compound of the invention in conjunction with the other therapeutic agent, and may thus be presented either as separate formulations, wherein at least one of those formulations comprises compound of the invention and at least one comprises the other therapeutic agent, or may be presented (i.e. formulated) as a combined preparation (i.e. presented as a single formulation including compound of the invention and the other therapeutic agent).

Thus, there is further provided:

(1) a pharmaceutical formulation including a compound of formula I, as hereinbefore defined but without the provisos, or a pharmaceutically-acceptable salt thereof, another therapeutic agent that is useful in the treatment of inflammation, and a pharmaceutically-acceptable adjuvant, diluent or carrier; and
(2) a kit of parts comprising components:
(a) a pharmaceutical formulation including a compound of formula I, as hereinbefore defined but without the provisos, or a pharmaceutically-acceptable salt thereof, in admixture with a pharmaceutically-acceptable adjuvant, diluent or carrier; and
(b) a pharmaceutical formulation including another therapeutic agent that is useful in the treatment of inflammation in admixture with a pharmaceutically-acceptable adjuvant, diluent or carrier,
   which components (a) and (b) are each provided in a form that is suitable for administration in conjunction with the other.

The invention further provides a process for the preparation of a combination product as hereinbefore defined, which process comprises bringing into association a compound of formula I, as hereinbefore defined but without the provisos, or a pharmaceutically acceptable salt thereof with the other therapeutic agent that is useful in the treatment of inflammation, and at least one pharmaceutically-acceptable adjuvant, diluent or carrier.

By “bringing into association”, we mean that the two components are rendered suitable for administration in conjunction with each other.

Thus, in relation to the process for the preparation of a kit of parts as hereinbefore defined, by bringing the two components “into association with” each other, we include that the two components of the kit of parts may be:

(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Compounds of the invention may be administered at varying doses. Oral, pulmonary and topical dosages may range from between about 0.01 mg/kg of body weight per day (mg/kg/day) to about 100 mg/kg/day, preferably about 0.01 to about 10 mg/kg/day, and more preferably about 0.1 to about 5.0 mg/kg/day. For e.g. oral administration, the compositions typically contain between about 0.01 mg to about 500 mg, and preferably between about 1 mg to about 100 mg, of the active ingredient. Intravenously, preferred doses will range from about 0.001 to about 10 mg/kg/hour during constant rate infusion. Advantageously, compounds may be administered in a single daily dose, or the total daily dosage may be administered in divided doses of two, three or four times daily.

In any event, the physician, or the skilled person, will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

Compounds of the invention may have the advantage that they are effective and/or selective inhibitors of lipoxygenases, and particularly 15-lipoxygenase.

Compounds of the invention may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the stated indications or otherwise.

### Biological Test

The assay employed takes advantage of the ability of lipoxygenases to oxidize polyunsaturated fatty acids, containing a 1,4-cis-pentadiene configuration, to their corresponding hydroperoxy or hydroxyl derivatives. In this particular assay, the lipoxygenase was a purified human 15-lipoxygenase and the fatty acid was arachidonic acid. The assay is performed at room temperature (20-22° C.) and the following are added to each well in a 96-well microtiter plate:

a) 35 μL phosphate buffered saline (PBS) (pH 7.4);
b) inhibitor (i.e. compound) or vehicle (0.5 μl DMSO);
c) 10 μL of a 10× concentrated solution of 15-lipoxygenase in PBS. The plates are incubated for 5 minutes at room temperature;
d) 5 μl of 0.125 mM arachidonic acid in PBS. The plate is then incubated for 10 minutes at room temperature;
e) the enzymatic reaction is terminated by the addition of 100 μl methanol; and
f) the amount of 15-hydroperoxy-eicosatetraenoic acid or 15-hydroxy-eicosatetraenoic acid is measured by reverse phase HPLC.

The invention is illustrated by way of the following examples, in which the following abbreviations may be employed:

aq. aqueous
BuLi n-butyllithium
DMAP 4-dimethylaminopyridine
DMF dimethylformamide
DIPEA diisopropylethylamine
EtOAc ethyl acetate
EtOH ethanol
MeOH methanol
MS mass spectrum
NMR nuclear magnetic resonance
PCA pyrazole-3-carboxylic acid
rt room temperature
sat. saturated
TBTU O-benzotriazol-1-yl-N,N,N′,N′-tetramethyluronium tetrafluoroborate
THF tetrahydrofuran

Chemicals specified in the synthesis of the compounds in the examples were commercially available from, e.g. Sigma-Aldrich Fine Chemicals.

Unless otherwise stated, one or more tautomeric forms of compounds of the examples described hereinafter may be prepared in situ and/or isolated. All tautomeric forms of compounds of the examples described hereinafter should be considered to be disclosed.

### Synthesis of Intermediates

### Dipyrazolo[1,5-a;1′,5′-d]pyrazine-4,9-dione (I)

SOCl₂ (20 mL) was added to a solution of pyrazole-3-carboxylic acid (5.0 g, 44.6 mmol) in THF (40 mL) at rt. The mixture was heated at reflux for 1 h and concentrated. The material was used without further purification.

### 5-Chloropyrazole-3-carboxylic Acid (II)

The title compound may be prepared from two alternative methods:

### Method A

### (a) 5-Chloro-3-methylpyrazole

A mixture of 5-chloro-1,3-dimethylpyrazole (2.6 mmol) and pyridine hydro-chloride (13.1 mmol) in a sealed 5 mL process vial was heated using microwave irradiation for 2 h at 200° C. After cooling to rt, EtOAc (15 mL) was added and the mixture was washed with HCl (aq., 2M; 10 mL), NaCl (sat., aq.), dried (MgSO₄) and concentrated to afford the sub-title compound as a white solid (Yield: 210 mg (67%)).

MS (M⁺+H) m/z=117.

¹H-NMR (DMSO-d₆, 400 MHz), δ 12.66 (br s, 1H), 6.03 (m, 1H), 2.19 (s, 3H).

### (b) 5-Chloropyrazole-3-carboxylic Acid

A mixture of 5-chloro-3-methylpyrazole (3.6 mmol; see step (a) above), water (6 mL) and tert-butanol (1.2 mL) was heated to 75° C., after which KMnO₄ (1.42 g, 9 mmol) was added. The mixture was stirred at 75° C. overnight and filtered hot. The solids were washed with boiling water. The combined cooled filtrates were extracted with EtOAc, and the combined extracts washed with NaCl (sat., aq.), dried (MgSO₄) and concentrated. The crude solid was recrystallised from EtOAc/hexane/pentane to give the sub-title compound as white crystals (Yield: 350 mg (67%)).

¹H-NMR (DMSO-d₆, 400 MHz), δ 13.65 (br s, 1H), 6.80 (s, 1H).

### Method B

### (a) 1-Benzenesulfonyl-3-methylpyrazole

A mixture of 3-methylpyrazole (5 g, 60.9 mmol), benzenesulfonyl chloride (8.55 mL, 67 mmol) and triethylamine (9.3 mL, 67 mmol) in acetonitrile was heated at reflux for 2 h, allowed to cool and concentrated. EtOAc (300 mL) was added and the solution was filtered and concentrated to provide a solid residue which was crystallised from EtOAc to give the title compound as an off-white powder (Yield: 7.92 g, 58%).

¹H-NMR (DMSO-d₆): δ 8.35 (d, 1H), 7.97-7.94 (m, 2H), 7.78 (tt, 1H), 7.66 (t, 2H), 6.43 (d, 1H), 2.17 (s, 3H).

### (b) 5-Chloro-1-(2-chlorobenzenesulfonyl)-3-methylpyrazole

BuLi (1.6M, 5.9 mL, 9.45 mmol) was added under argon to a solution of 1-benzenesulfonyl-3-methylpyrazole (940 mg, 4.5 mmol; see step (a) above) in THF (50 mL) at −78° C. The mixture was stirred for approximately 30 min before hexachloroethane (3.7 g, 15.8 mmol) was added. After stirring at −78° C. for 18 h, NH₄Cl (sat., aq.; 50 mL) was added and the mixture was allowed to come to rt. Water (50 mL) was added, the layers separated, and the aqueous phase extracted with EtOAc (2×100 mL). The combined organic phases were dried (Na₂SO₄) and concentrated. Purification by chromatography (1:4 EtOAc/heptane), followed by recrystallisation from EtOAc/heptane, gave the title compound as white crystals (Yield: 1.1 g, 84%).

¹H-NMR (DMSO-d₆): δ 8.17 (dd, 1H), 7.87-7.67 (m, 4H), 2.15 (s, 3H).

### (c) 5-Chloro-3-methylpyrazole

Sodium ethoxide (2.5M, 16.1 mL, 40.3 mmol) was added to a solution of 5-chloro-1-(2-chlorobenzenesulfonyl)-3-methylpyrazole (6.9 g, 27 mmol; see step (b) above) dissolved in EtOH (50 mL). The solution was stirred for 30 min at rt, water (100 mL) was added, the mixture was neutralised using HCl (aq., 2M) and extracted with EtOAc (3×100 mL). Concentration of the combined organic phases resulted in precipitation prior to complete solvent removal. The precipitate was filtered off and the filtrate was concentrated to give the title compound as a brown oil that crystallised on standing (Yield: 1.0 g, 33%) which was used without further purification.

¹H-NMR (DMSO-d₆): δ 12.66 (br s, 1H), 6.03 (d, 1H), 2.20 (s, 3H).

### (d) 5-Chloropyrazole-3-carboxylic Acid

A solution of KMnO₄ (3.5 g, 22 mmol) in water (120 mL) was added in portions over a period of 5 h at 70° C. to a solution of 5-chloro-3-methylpyrazole (1.0 g, 8.8 mmol; see step (c) above) in water (50 mL) and tert-butanol (1 mL). The mixture was stirred at 70° C. overnight and filtered through Celite®. The colourless filtrate was concentrated and acidified with HCl (aq., 2M). Filtration gave the title compound as a white powder which was used without further purification. (Yield: 913 mg, 80%).

¹H-NMR (DMSO-d₆): δ 6.80 (s, 1H), 4.40 (br s, 1H).

### 2,7-Dichlorodipyrazolo[1,5-a;1′,5′-d]pyrazine-4,9-dione (III)

A mixture of 5-chloropyrazole-3-carboxylic acid (80 mg; see step (b) above) and SOCl₂ (1 mL) was heated at 80° C. for 18 h. The excess SOCl₂ was removed in vacuo and the crude product (69 mg) was used without purification.

### 4,5-Dichloropyrazole-3-carboxylic Acid (IV)

Chlorine gas was bubbled slowly through a stirred solution of 5-chloropyrazole-3-carboxylic acid (Intermediate II, 3.00 g, 20.5 mmol) in water (2.0 L) at rt over 3 h. The solution was stirred for 18 h in an open flask and then concentrated in vacuo. The slurry was extracted with ethyl acetate (3×100 mL), the combined extracts were washed with NaCl (sat., aq.; 100 mL) and dried (Na₂SO₄). The solvent was removed in vacuo to give the product as a white powder. Yield 3.20 g (86%).

MS (M⁻−H) m/z=179.

¹H NMR (DMSO-d₆, 400 MHz) δ 14.44 (s, 1H), 14.09 (s, 1H).

¹³C NMR (CD₃OD, 100 MHz) δ 160.0; 139.6; 133.1; 112.4.

### 4-Chloro-5-trifluoromethylpyrazole-3-carboxylic Acid (V)

KMnO₄ (10.7 g, 67.7 mmol) was added portion-wise to a mixture of 5-trifluoromethyl-4-chloro-3-methylpyrazole (5.0 g, 27.1 mmol), t-BuOH (50 mL) and water (250 mL). The mixture was stirred at 75° C. for 3 days. The mixture was allowed to cool to rt, filtered and concentrated. HCl (sat., aq.; 10 mL) was added and the mixture extracted with EtOAc (5×30 mL). The combined extracts were washed with NaCl (sat., aq.; 50 mL), dried (Na₂SO₄) and concentrated to give the product (4.90 g, 84%) as a white solid.

MS (M⁻−H) m/z=213.

### 1-Diethylcarbamoyl-5-difluoromethylpyrazole-3-carboxylic Acid Ethyl Ester (VI)

### (a) Copper(II) 5-ethoxy-1,1-difluoro-4,5-dioxopent-2-en-2-olate

1,1-Difluoroacetone (1.0 g, 10.63 mmol) was added dropwise to a stirred mixture of lithium hydride (85 mg, 10.63 mmol) and diethyl oxalate (1.55 g, 10.63 mmol) in dry diethyl ether (10 mL). The mixture was heated to reflux for 18 h. After cooling to rt, a solution of acetic acid (638 μL, 11.16 mmol) in water (2 mL) and a suspension of copper(II) acetate (2.23 g, 11.16 mmol) in water (8 mL) were subsequently added to the reaction mixture. The mixture was stirred at rt for 5 min, the precipitate formed was filtered off and dried in the air. Yield: 3.72 g (77%) of dark blue powder.

m.p.: 145.6-147.9° C.

### (b) 5-Difluoromethylpyrazole-3-carboxylic Acid Ethyl Ester

Hydrazine monohydrate (570 μL, 11.7 mmol) followed by HCl (2.7 mL of 4.3M solution in EtOH; 11.7 mmol) were added to a solution of copper(II) 5-ethoxy-1,1-difluoro-4,5-dioxopent-2-en-2-olate (2.4 g, 10.62 mmol) in EtOH (15 mL) at 0° C. The mixture was stirred at rt for 16 h followed by heating at reflux for 3 h. The mixture was concentrated and the residue purified by chromatography (heptane/EtOAc, 3:1) to give the sub-title compound as white crystals. Yield 795 mg (40%).

¹H NMR (DMSO-d₆, 400 MHz) δ14.45 (br. s, 1H), 7.04 (s, 1H), 7.04 (t, 1H), 4.33 (q, 2H), 1.32 (t, 3H).

### (c) 1-Diethylcarbamoyl-5-difluoromethylpyrazole-3-carboxylic Acid Ethyl Ester

1,4-Diazabicyclo[2.2.2]octane (98 mg, 0.87 mmol) was added to a suspension of 5-difluoromethylpyrazole-3-carboxylic acid ethyl ester (150 mg, 0.79 mmol) in CH₃CN (4.0 mL). After stirring at rt for 15 minutes diethylcarbamoyl chloride (118 mg, 0.87 mmol) was added. The solution was stirred at rt for 1 h, water (30 mL) was added and the mixture was extracted with EtOAc (3×40 mL). The combined organic phases were washed with brine (50 mL), dried (Na₂SO₄) and concentrated to give the title compound as yellow oil. Yield: 204 mg (89%).

¹H NMR (DMSO-d₆, 400 MHz) δ 7.32 (t, 1H), 7.21 (s, 1H), 4.33 (q, 2H), 3.46 (br. s, 4H), 1.31 (t, 3H), 1.19 (br. s, 6H).

### 4-Chloropyrazole-3-carboxylic Acid (VII)

### (a) 4-Chloro-3-methylpyrazole Hydrochloride

A stirred solution of 3-methylpyrazole (50 mmol, 4.10 g) in carbon tetrachloride (50 mL) was saturated with chlorine gas at −78° C. The temperature was allowed to rise to rt and the mixture was stirred overnight. The slurry was diluted with pentane (50 mL) and stirred for 30 min. The white crystalline solid was filtered off, washed with pentane (2×50 mL) and dried. Yield 7.50 g (98%).

MS (M⁺+H) m/z=117.

¹H NMR (DMSO-d₆, 400 MHz) δ 13.38 (s, 2H), 7.68 (s, 1H), 2.16 (s, 3H).

¹³C NMR (DMSO-d₆, 100 MHz) δ 139.1, 132.2, 106.8, 9.3.

### (b) 4-Chloropyrazole-3-carboxylic Acid

A well-stirred mixture of 4-chloro-3-methylpyrazole hydrochloride (20 mmol, 3.06 g) and potassium permanganate (50 mmol, 11.4 g) in water (500 mL) was stirred for 3 days at rt and then for 5 h at 70° C. The solids were filtered off and the colorless filtrate was concentrated. HCl (aq., 1M; 50 mL) was added and the mixture extracted with EtOAc (5×50 mL). The combined organic extracts were washed with NaCl (sat., aq.), dried (Na₂SO₄) and concentrated in vacuo to give 640 mg (22%) of the sub-title compound as a white solid.

¹H NMR (DMSO-d₆, 400 MHz) δ 13.47 (br. s, 2H), 7.92 (br. s, 1H).

### 1-Diethylcarbamoyl-5-methyl-4-trifluoromethylpyrazole-3-carboxylic Acid Ethyl Ester (VIII)

### (a) 2-Diazo-4,4,4-trifluoro-3-oxobutyric Acid Ethyl Ester

A solution of trifluoroacetic anhydride (15.3 mL, 110 mmol) in CH₂Cl₂ (25 mL) was added drop-wise over 25 min to a solution of diazoacetic acid ethyl ester (10.5 mL, 100 mmol) and pyridine (8.9 mL, 110 mmol) in CH₂Cl₂ (100 mL) under argon at 0° C. The mixture was poured into water (100 mL) and neutralised with. NaHCO₃ (sat. aq.; 200 mL). The organic phase was washed with CuSO₄ (aq., 1M, 3×50 mL) and the combined aqueous layers were extracted with CH₂Cl₂ (2×25 mL). The combined extracts were washed with NaCl (sat., aq.; 20 mL), dried (Na₂SO₄) and concentrated to give a dark oil that was used without further purification. Yield: 18.82 g (90%).

¹H NMR (DMSO-d₆, 400 MHz) δ 4.28 (q, 2H), 1.26 (t, 3H).

¹³C NMR (DMSO-d₆, 400 MHz) δ 171.3 (q), 162.7, 115.6 (q), 76.9, 61.9, 13.7

### (b) N,N-Diethylprop-1-ynylamine

N,N-Diethylpropargylamine (21 mL, 16.8 g, 0.151 mol) was added in one portion to a solution of potassium tert-butoxide (0.72 g, 6.4 mmol) in DMSO (20 mL). The solution was cooled occasionally with an ice/water bath to keep the temperature below 50°. After 40 min, the reaction mixture was quickly vacuum distilled through a 15 cm Vigreux column with a single receiver cooled to −10° C. The distillate was collected until a few ml of DMSO (˜74° C. at 15 mbar) had distilled. The collected distillate was heated to 120° under argon for 30 min, followed by another vacuum distillation. The product was collected as a clear colorless liquid, boiling at 28-32° C. (15-16 mbar). Yield: 12.7 g (76%).

¹H NMR (DMSO-d₆, 400 MHz) δ 2.78 (q, 4H), 1.80 (s, 3H), 1.08 (t, 6H).

### (c) 1-Diethylcarbamoyl-5-methyl-4-trifluoromethylpyrazole-3-carboxylic Acid Ethyl Ester

A solution of N,N-diethylprop-1-ynylamine (0.83 mL, 0.67 g, 6.0 mmol) in diethyl ether (5 mL) was added dropwise over 5 min to a solution of 2-diazo-4,4,4-trifluoro-3-oxobutyric acid ethyl ester (1.07 g, 5.09 mmol) in diethyl ether (10 mL). After stirring for 4 h at rt, the reaction mixture was concentrated. The residue was purified by chromatography (EtOAc/heptane, 80:20 to 60:40) to give the product as clear yellow oil. Yield: 0.84 g (51%).

¹H NMR (DMSO-d₆, 400 MHz) δ 4.34 (q, 2H), 3.50 (q, 2H), 3.14 (q, 2H), 2.46 (s, 3H), 1.29 (t, 3H), 1.22 (t, 3H), 1.11 (t, 3H).

¹³C NMR (DMSO-d₆, 400 MHz) δ 159.7, 149.5, 142.6, 140.5, 122.0 (q), 110.2 (q), 61.3, 43.6, 42.0, 13.8, 13.5, 12.2, 10.5.

### 4,5-Bis(trifluoromethyl)pyrazole-3-carboxylic Acid (IX)

### (a) 2,3-Bis(trifluoromethyl)pyrazolo[1,5-a]pyridine

A mixture of 1-aminopyridinium iodide (3.00 g, 13.51 mmol), K₂CO₃ (3.73 g, 27.02 mmol) and 2,3-dichloro-1,1,1,4,4,4-hexafluoro-but-2-ene (mixture of cis and trans, 9.18 g, 39.41 mmol) were stirred in THF (100 mL) at rt for 24 h. The mixture was partitioned between EtOAc (100 mL), water (100 mL) and HCl (aq., 2M; 5 mL). The phases were separated and the organic phase washed with NaCl (sat., aq.; 50 mL), dried (Na₂SO₄) and concentrated. The residue was dissolved in MeOH (25 mL) and filtered through Celite®. The filtrate was concentrated to give the title compound as slightly yellow needles. Yield: 2.95 g (86%).

¹H NMR (CDCl₃, 400 MHz) δ 8.45 (ddd, 1H), 7.75 (dd, 1H), 7.39 (ddd, 1H), 7.02 (ddd, 1H).

### (b) 4,5-Bis(trifluoromethyl)pyrazole-3-carboxylic Acid

KMnO₄ (7.74 g, 49.0 mmol) was added portion-wise to a mixture of 2,3-bis(trifluoromethyl)pyrazolo[1,5-a]pyridine (2.49 g, 9.80 mmol), t-BuOH (30 mL) and water (120 mL). After stirring at rt for 24 h the mixture was filtered through Celite®. The filtrate was washed with CH₂Cl₂ (2×50 mL), then pH was adjusted to 1 with HCl (conc. aq.) and the mixture was concentrated. The residue was extracted with acetone (3×20 mL), the combined extracts were concentrated and the residue was crystallised from HCl (aq., 0.1M; 2.5 mL). The solid was collected, washed with water (2×0.5 mL) and dried in vacuo to give the title compound as a white solid. Yield: 1.63 g (67%).

¹³C NMR (CD₃OD, 100 MHz) δ 159.2 (s), 141.3 (q), 137.7 (s), 122.4 (q), 121.6 (q), 112.4 (q).

### 4-Chloro-5-methylpyrazole-3-carboxylic Acid Ethyl Ester (X)

N-Chlorosuccinimide (1.17 g, 8.76 mmol) was added portion-wise to a solution of 5-methylpyrazole-3-carboxylic acid ethyl ester (900 mg, 5.84 mmol) in CH₂Cl₂ (10 mL) at 0° C. The mixture was allowed to warm to rt and stirred at rt for 19 h. The mixture was washed with saturated, aqueous Na₂CO₃ (1:1, 4×30 mL), the organic phase dried (Na₂SO₄) and concentrated to give the title compound as yellow-green solid. Yield: 846 mg (77%).

¹H NMR (DMSO-d₆, 400 MHz) δ 13.68 (br. s, 1H), 4.29 (q, 2H), 2.21 (s, 3H), 1.29 (t, 3H).

### 5-Difluoromethyl-4-chloropyrazole-3-carboxylic acid (XI)

### (a) 5-Difluoromethylpyrazole-3-carboxylic Acid

KMnO₄ (2.74 g, 9.45 mmol) was added in portions to a mixture of 5-difluoromethyl-3-methylpyrazole (500 mg, 3.78 mmol), t-BuOH (10 mL) and water (100 mL). The mixture was stirred at 75° C. for 18 h. After cooling to rt the precipitate (MnO₂) was filtered off and the filtrate was concentrated in vacuo. HCl (aq., conc.; 2.0 mL) was added and the mixture was extracted with EtOAc (5×20 mL). The combined extracts were washed with NaCl (sat., aq.; 25 mL), dried (Na₂SO₄) and concentrated. The material was purified using reverse phase column (RP-18) and CH₃CN/water (1:2) as eluent (Yield: 250 mg, %).

MS (M⁻−H) m/z=161.

¹H NMR (DMSO-d₆, 400 MHz) δ 14.27 (s, 1H), 13.60 (br. s, 1H), 7.03 (t, 1H), 6.91 (s, 1H).

### (b) 5-Difluoromethyl-4-chloropyrazole-3-carboxylic Acid

Chlorine gas was bubbled slowly through a stirred solution of 5-difluoro-methylpyrazole-3-carboxylic acid (100 mg, 0.62 mmol) in water (100 mL) at rt over 3 h. The solution was stirred for 18 h in an open flask and concentrated. The slurry was extracted with EtOAc (3×20 mL), the combined organic phases were washed with NaCl (sat., aq., 25 mL), dried (Na₂SO₄) and concentrated in vacuo to give the product as a white powder (Yield 106 mg, 87%).

MS (M⁻−H) m/z=195, 197.

### Synthesis of arylamines

Arylamines, which were not available commercially, were synthesised according to the procedures described below.

### 2-Amino-5,6-dimethoxypyridine

### (a) 2-Bromo-3-methoxy-6-nitropyridine

2-Bromo-3-methoxypyridine (4.45 g, 23.7 mmol) was added to a mixture of fuming HNO₃ and concentrated H₂SO₄ (1:1, 18 mL) at 0° C. The mixture was stirred at 55° C. for 1.5 h and then poured into ice water (150 mL). The precipitate formed was the pure product, which was used without further purification. Yield: 3.54 g (64%) of slightly yellow solid.

¹H NMR (DMSO-d₆, 400 MHz) δ 8.41 (d, 1H), 7.80 (d, 1H), 4.06 (s, 3H).

### (b) 2,3-Dimethoxy-6-nitropyridine

Sodium methoxide (927 μL of 30% solution in methanol, 5.2 mmol) was added to a mixture of 2-bromo-3-methoxy-6-nitropyridine (750 mg, 3.22 mmol), DMSO (6 mL) and MeOH (9 mL). The mixture was stirred at rt for 90 min, at 35° C. for 24 h and at rt for 24 h. The mixture was poured into ice water (150 mL). The solid was filtered off, washed with water (100 mL) and dried in vacuo. Yield: 453 mg (76%) of slightly yellow solid.

¹H NMR (DMSO-d₆, 400 MHz) δ 8.02 (d, 1H), 7.55 (d, 1H), 3.97 (s, 3H), 3.94 (s, 3H).

### (c) 2-Amino-5,6-dimethoxypyridine

2,3-Dimethoxy-6-nitropyridine (450 mg, 2.44 mmol) in MeOH (10 mL) and CH₂Cl₂ (10 mL) was hydrogenated over Pd on carbon (10%, 100 mg) at ambient temperature and pressure for 3 h. The mixture was filtered through Celite® and the filtrate was concentrated in vacuo to give the title product as a light brown solid.

Yield: 356 mg (95%).

¹H NMR (DMSO-d₆, 400 MHz) δ 7.05 (d, 1H), 5.92 (d, 1H), 5.36 (br. s, 2H), 3.75 (s, 3H), 3.60 (s, 3H).

### 2-Amino-5-methoxypyridine

A mixture of 2-bromo-3-methoxy-6-nitropyridine (1.20 g, 5.15 mmol), hydrazine hydrate (6 mL), Pd on carbon (10%, 400 mg) in ethanol (40 mL) was heated at reflux for 45 min. The mixture was allowed to cool, filtered through Celite® and concentrated. Water (20 mL) and NH₃ (sat., aq.; 10 mL) were added and the mixture extracted with chloroform (2×50 mL). The combined organic extracts were dried (Na₂SO₄) and concentrated to give the title product as a low melting colourless solid. Yield: 615 mg (96%).

¹H NMR (DMSO-d₆, 400 MHz) δ 7.64 (dd, 1H), 7.10 (dd, 1H), 6.42 (dd, 1H), 5.43 (br. s, 2H), 3.68 (s, 3H).

### 2-Amino-3-methoxypyridine

Prepared by a procedure analogous to described above for 2-amino-5,6-dimethoxypyridine, step (c), from 3-methoxy-2-nitropyridine (1.598 g, 10.4 mmol). Yield: 961 mg (74%) of white needles.

¹H NMR (DMSO-d₆, 400 MHz) δ 7.49 (dd, 1H), 6.99 (dd, 1H), 6.49 (dd, 1H), 5.60 (br. s, 2H), 3.76 (s, 3H).

### 2-Amino-5-ethoxypyridine

### (a) 2-Bromo-3-ethoxypyridine

A mixture of 2-bromopyridin-3-ol (2.00 g, 11.5 mmol), iodoethane (3.12 g, 20 mmol) and K₂CO₃ (2.49 g, 18 mmol) in DMF (17 mL) was stirred at 80° C. for 110 min. The mixture was concentrated and the residue was partitioned between EtOAc (100 mL) and water (50 mL). The aqueous phase was extracted with EtOAc (50 mL), the combined organic phases were washed with water (25 mL) and NaCl (sat., aq.; 25 mL), dried (Na₂SO₄) and concentrated to give the sub-title compound as brown oil. Yield: 2.15 g (92%).

¹H NMR (DMSO-d₆, 400 MHz) δ 7.95 (dd, 1H), 7.51 (dd, 1H), 7.39 (dd, 1H), 4.15 (q, 2H), 1.36 (t, 3H).

### (b) 2-Bromo-3-ethoxy-6-nitropyridine

Prepared by a procedure analogous to the one described above for 2-bromo-3-methoxy-6-nitropyridine from 2-bromo-3-ethoxypyridine (1.827 g, 9.04 mmol).

Yield: 1.53 g (68%) of slightly yellow solid.

¹H NMR (DMSO-d₆, 400 MHz) δ 8.39 (d, 1H), 7.79 (d, 1H), 4.33 (q, 2H), 1.42 (t, 3H).

### (c) 2-Amino-5-ethoxypyridine

Prepared by a procedure analogous to the one described above for 2-amino-5-methoxypyridine from 2-bromo-3-ethoxy-6-nitropyridine (1.50 g, 6.08 mmol).

Yield: 836 mg (100%) of yellow oil.

¹H NMR (DMSO-d₆, 400 MHz) δ 7.62 (d, 1H), 7.09 (dd, 1H), 6.40 (d, 1H), 5.42 (br. s, 2H), 3.91 (q, 2H), 1.26 (t, 3H).

### 2-Amino-5-propoxypyridine

### (a) 2-Bromo-3-propoxypyridine

Prepared by a procedure analogous to the one described above for 2-bromo-3-ethoxypyridine from 2-bromopyridin-3-ol (2.00 g, 11.5 mmol) and 1-iodopropane.

Yield: 2.26 g (91%) of light-brown oil.

¹H NMR (DMSO-d₆, 400 MHz) δ 7.95 (dd, 1H), 7.51 (dd, 1H), 7.39 (dd, 1H), 4.06 (t, 2H), 1.82-1.70 (m, 2H), 1.01 (t, 3H).

### (b) 2-Bromo-6-nitro-3-propoxypyridine

Prepared by a procedure analogous to the one described above for 2-bromo-3-methoxy-6-nitropyridine from 2-bromo-3-propoxypyridine (2.20 g, 10.2 mmol).

Yield: 1.58 g (59%) of slightly yellow solid.

¹H NMR (DMSO-d₆, 400 Mhz) δ 8.38 (d, 1H), 7.79 (d, 1H), 4.23 (t, 2H), 1.87-1.75 (m, 2H), 1.02 (t, 3H).

### (c) 2-Amino-5-propoxypyridine

Prepared by a procedure analogous to the one described above for 2-amino-5-methoxypyridine from 2-bromo-6-nitro-3-propoxypyridine (1.55 g, 5.94 mmol).

Yield: 913 mg (100%) of white solid.

¹H NMR (DMSO-d₆, 400 MHz) δ 7.62 (d, 1H), 7.09 (dd, 1H), 6.40 (d, 1H), 5.41 (br. s, 2H), 3.81 (t, 2H), 1.71-1.59 (m, 2H), 0.94 (t, 3H).

### 2-Amino-5-butoxypyridine

### (a) 2-Bromo-3-butoxypyridine

Prepared by a procedure analogous to the one described above for 2-bromo-3-ethoxypyridine from 2-bromopyridin-3-ol (1.74 g, 10.0 mmol) and 1-iodobutane.

Yield: 2.185 g (95%) of yellow oil

¹H NMR (DMSO-d₆, 400 MHz) δ 7.94 (dd, 1H), 7.51 (dd, 1H), 7.39 (dd, 1H), 4.06 (t, 2H), 1.77-1.68 (m, 2H), 1.53-1.40 (m, 2H), 0.94 (t, 3H)

### (b) 2-Bromo-3-butoxy-6-nitropyridine

Prepared by a procedure analogous to the one described above for 2-bromo-3-methoxy-6-nitropyridine from 2-bromo-3-butoxypyridine (2.10 g, 9.13 mmol).

Yield: 1.04 g (41%) of slightly yellow solid.

¹H NMR (DMSO-d₆, 400 MHz): δ 8.39 (d, 1H), 7.80 (d, 1H), 4.28 (t, 2H), 1.82-1.67 (m, 2H), 1.54-1.42 (m, 2H), 0.96 (t, 3H).

### (c) 2-Amino-5-butoxypyridine

Prepared by a procedure analogous to the one described above for 2-amino-5-methoxypyridine from 2-bromo-3-butoxy-6-nitropyridine (1.03 g, 3.74 mmol).

Yield: 501 mg (81%) of white solid.

¹H NMR (DMSO-d₆, 400 Mhz) δ 7.63 (d, 1H), 7.09 (dd, 1H), 6.41 (d, 1H), 5.42 (br. s, 2H), 3.81 (t, 2H), 1.68-1.58 (m, 2H), 1.47-1.34 (m, 2H), 0.92 (t, 3H).

### 2-Amino-5-ethylpyridine

Diethylzinc (24 mL of 1M solution in hexane; 24 mmol) was added dropwise to a solution of 2-amino-5-bromopyridine (2.0 g, 11.6 mmol) and Pd(dppf)Cl₂.CH₂Cl₂ (225 mg, 0.28 mmol) in degassed dioxane (45 mL). The mixture was stirred at rt for 2 h, heated at reflux for 3 h and stirred at rt for 70 h under argon. The mixture was poured into NaCl (sat., aq.; 150 mL) and extracted with EtOAc (4×100 mL). The combined organic phases were washed with NaCl (sat., aq.; 100 mL), dried (Na₂SO₄) and concentrated. The residue was purified by chromatography (EtOAc/heptane, then MeOH/EtOAc) to give the title compound. Yield: 1.40 g (99%).

¹H NMR (DMSO-d₆, 400 MHz) δ 7.74 (s, 1H), 7.25 (dd, 1H), 6.40 (d, 1H), 5.67 (br. s, 2H), 2.39 (q, 2H), 1.10 (t, 3H).

### 2-Amino-5-propylpyridine

Propylmagnesiumbromide (6 mL of 2M solution in diethyl ether; 12 mmol) was added to a solution of zinc chloride (6 mL of 1M solution in diethyl ether; 6 mmol) under argon at 0° C. The solution was diluted with dioxane (10 mL) and transferred into a suspension of 2-amino-5-bromopyridine (516 mg, 3 mmol) and Pd(dppf)Cl₂.CH₂Cl₂ (55 mg, 0.07 mmol) in dioxane (5 mL). The mixture was heated at reflux for 20 h. After cooling to rt the mixture was poured into water (50 mL) and NaHCO₃ (aq., 1M; 20 mL) was added. The mixture was extracted with EtOAc (3×50 mL). The combined organic phases were washed with brine (50 mL), dried (Na₂SO₄) and concentrated to give 575 mg of a dark oil, which was used without further purification.

¹H NMR (CD₃OD, 400 MHz) δ 7.74 (d, 1H), 7.43 (d, 1H), 6.62 (d, 1H), 2.43 (t, 2H), 1.55-1.62 (m, 2H), 0.91 (t 3H).

### 2-Amino-5-butylpyridine

Prepared by a procedure analogous to the one described above for 2-amino-5-propylpyridine from 2-amino-5-bromopyridine (1.30 g, 6 mmol) and butylmagnesiumchloride (12 mL of 2M solution in THF, 24 mmol). The crude product was purified by column chromatography (EtOAc/heptane) to give 405 mg (45%) of the title compound as brown solid.

¹H NMR (DMSO-d₆, 400 MHz) δ 7.71 (d, 1H), 7.21 (dd, 1H), 6.37 (d, 1H), 5.61 (br. s, 2H), 2.37 (t, 1H), 1.46 (p, 2H), 1.25-1.30 (m, 2H), 0.88 (t, 3H).

### 2-Amino-5-ethyl-6-methylpyridine

Prepared by a procedure analogous to the one described above for 2-amino-5-ethylpyridine from 2-amino-5-bromo-6-methylpyridine (2.0 g, 10.7 mmol). Purification by chromatography (EtOAc/heptane) gave the title compound as brown crystals. Yield: 0.74 g (51%).

¹H NMR (DMSO-d₆, 400 MHz) δ 7.06 (d, 1H), 6.21 (d, 1H), 5.51 (s, 2H), 2.40 (q, 2H), 2.21 (s, 3H), 1.06 (t, 3H).

### 2-Amino-5,6-dimethylpyridine

A mixture of 2-amino-5-bromo-6-methylpyridine (561 mg, 3.0 mmol), K₂CO₃ (1.24 g, 9.0 mmol) and Pd(dppf)Cl₂.CH₂Cl₂ (245 mg, 0.30 mmol) was added to a solution of trimethylboroxine (377 mg, 3.0 mmol) in water (1 mL) and dioxane (10 mL). The mixture was heated at reflux for 3 h. After cooling to rt, the mixture was poured into water (50 mL). The mixture was extracted with diethyl ether (3×50 ml), the combined organic phases were dried (Na₂SO₄) and concentrated. Purification by chromatography (EtOAc/heptane) gave the title compound as black-brown solid. Yield: 244 mg (67%).

¹H NMR (DMSO-d₆, 400 MHz) δ 7.09 (d, 1H), 6.18 (d, 1H), 5.50 (br. s, 2H), 2.18 (s, 3H), 2.03 (s, 3H).

### 2-Amino-5-(tert-butyldimethylsilyloxymethyl)pyridine Hydrochloride

A mixture of 6-amino-3-(hydroxymethyl)pyridine (80 mg, 0.64 mmol), chloro-(tert-butyl)dimethylsilane (107 mg, 0.71 mmol) and DMAP (4.0 mg, 0.032 mmol) in DMF (1 mL) was stirred at rt for 24 h. The mixture was diluted with toluene (10 mL) and the precipitate formed was filtered off, washed with toluene (10 mL) and dried in vacuo. Yield: 121 mg (69%) of white plates.

¹H NMR (DMSO-d₆, 400 MHz) δ 13.78-13.45 (br. s, 1H), 8.04 (s, 2H), 7.9-7.8 (m, 2H), 7.01 (d, 1H), 4.58 (s, 2H), 0.89 (s, 9H), 0.09 (s, 6H).

### N-(6-Aminopyridin-3-yl)benzamide Hydrochloride

Benzoyl chloride (558 μL, 4.81 mmol) was added to a solution of pyridine-2,5-diamine (500 mg, 4.58 mmol) in THF (50 mL), and the mixture was stirred at rt for 1 h. The mixture was concentrated, the residue dissolved in hot MeOH/EtOAc (1:1) and the resulting mixture filtered hot. The filtrate was cooled to rt and concentrated. The residue was purified by chromatography (EtOAc/MeOH) to give the title compound as brown solid. Yield: 399 mg (35%).

¹H NMR (DMSO-d₆, 400 MHz) δ 14.16-13.52 (br. s, 1H), 19.68 (s, 1H), 8.56 (d, 1H), 8.23 (dd, 1H), 8.05-7.97 (m, 2H), 7.66-7.50 (m, 3H), 7.08 (d, 1H), 3.71-3.41 (br. s, 2H).

### EXAMPLES

### Example 1

### N-(5-Chloropyridin-2-yl)pyrazole-3-carboxamide

A mixture of intermediate (I) (188 mg, 1.00 mmol), 2-amino-5-chloropyridine (259 mg, 2.01 mmol), DMAP (245 mg, 2.01 mmol) and DMF (1 mL) was heated at 120° C. overnight. Concentration and purification by chromatography (EtOAc/MeOH) gave the title product as an off-white solid in (Yield: 141 mg (32%)).

¹H-NMR (DMSO-D₆): δ 13.55 (br s, 1H), 9.92 (br s, 1H), 8.41 (d, 1H), 8.21 (d, 1H), 7.99 (dd, 1H), 7.89 (br s, 1H), 6.94 (br s, 1H).

### Example 2

### 5-Chloro-N-(5-chloropyridin-2-yl)pyrazole-3-carboxamide

TBTU (1.2 mmol) was added to a solution of intermediate (II) (1.5 mmol), 2-amino-5-chloropyridine (1.8 mmol) and DIPEA (2.0 mmol) in dry DMF (2 mL). The mixture was stirred at 60° C. for 3 h and concentrated. Water was added and the mixture was extracted with EtOAc. The combined extracts were washed with CaCl₂ (sat., aq.), dried (Na₂SO₄) and concentrated. Crystallisation from EtOAc gave the title compound as a white powder (Yield: 170 mg (44%)).

¹H-NMR (DMSO-d₆): δ 14.06 (br s, 1H), 11.13 (br s, 1H), 8.55 (d, 1H), 8.19 (d, 1H), 7.98 (dd, 1H), 7.32 (s, 1H).

### Example 3

### 5-Chloro-N-(5-fluoropyridin-2-yl)pyrazole-3-carboxamide

A mixture of intermediate (III) (100 mg, 0.4 mmol), DMAP (95 mg, 0.8 mmol) and 2-amino-5-fluoropyridine (88 mg, 0.8 mmol) in CH₂Cl₂ (10 mL) was stirred at 60° C. for 18 h. The mixture was allowed to reach rt and concentrated. Water (10 mL) was added and the mixture was acidified to ca pH 4 with HCl (aq., 2M) and extracted with EtOAc (2×15 mL). The combined extracts were washed with NaCl (sat., aq.), dried (MgSO₄) and concentrated. Crystallisation from EtOH/water gave the title compound as a white powder (Yield: 38.9 mg, (42%)).

MS (M⁺+H) m/z=241

¹H-NMR (DMSO-d₆, 400 MHz), δ 11.08 (br s, 1H), 8.42 (d, 1H), 8.18 (dd, 1H), 7.80-7.85 (m, 1H), 7.33 (br s, 1H)

### Example 4

### N-(5-(trifluoromethyl)pyridin-2-yl)pyrazole-3-carboxamide

A mixture of intermediate (I) (188 mg, 1.0 mmol), DMAP (122 mg, 1.0 mmol) and 2-amino-5-(trifluoromethyl)pyridine (170 mg, 1.0 mmol) in DMF (7 mL) was stirred at 120° C. for 18 h and allowed to cool to rt. HCl (aq., 1M, 10 mL) was added and the mixture was extracted with EtOAc (2×20 mL). The combined extracts were washed with water, NaCl (sat., aq.), dried (MgSO₄) and concentrated. Crystallisation from EtOAc/pentane followed by crystallisation from EtOH/water furnished the title compound as a white powder (Yield: 95.3 mg, (35%)).

MS (M⁺+H) m/z=257

¹H-NMR (DMSO-d₆, 400 MHz), δ 13.64 (br s, 1H), 10.04 (br s, 1H), 8.75 (s, 1H), 8.37 (d, 1H), 8.24 (d, 1H), 7.93 (bs, 1H), 6.93 (br s, 1H)

### Example 5

### 5-Chloro-N-(5-(trifluoromethyl)pyridin-2-yl)pyrazole-3-carboxamide

A mixture of intermediate (III) (140 mg, 0.5 mmol), DMAP (133 mg, 1.0 mmol) and, 2-amino-5-trifluoromethylpyridine (186 mg, 1.0 mmol) in CH₂Cl₂ (15 mL) was stirred at 60° C. for 18 h and allowed to cool to rt. The solid was collected by filtration and washed with CH₂Cl₂. The solid was dissolved in EtOAc (20 mL) and washed with HCl (aq., 2M, 2×10 mL), NaCl (sat., aq.), dried (MgSO₄) and concentrated. Crystallisation from EtOH/water gave the title compound as white crystals (Yield: 81.7 mg, (52%)).

MS (M⁺+H) m/z=291

¹H-NMR (DMSO-d₆, 400 MHz), δ 14.14 (br s, 1H), 11.38 (br s, 1H), 8.80 (s, 1H), 8.26-8.80 (m, 2H), 7.37 (s, 1H)

### Example 6

### N-(5-bromo-6-methylpyridin-2-yl)-5-chloropyrazole-3-carboxamide

The title compound was prepared in accordance with Example 5 from intermediate (III) (140 mg, 0.5 mmol), DMAP (133 mg, 1.0 mmol) and 6-amino-3-bromo-2-methylpyridine (186 mg, 1.0 mmol). Yield: 32 mg (20%).

MS (M⁺+H) m/z=315

¹H-NMR (DMSO-d₆, 400 MHz), δ 14.14 (br s, 1H), 11.38 (br s, 1H), 8.03 (m, 2H), 7.39 (s, 1H), 2.47 (s, 3H)

### Example 7

### N-[6-(Pyrazole-3-carboxamido)pyridin-3-yl]pyrazole-3-carboxamide

A suspension of intermediate (I) (188 mg, 1.00 mmol) and 2,5-diaminopyridine (98 mg, 0.90 mmol) in pyridine (7 mL) and DMF (0.7 mL) was heated at 80° C. overnight. Concentration and purification by chromatography gave the title compound as an orange solid. Yield: 77 mg (52%).

¹H-NMR (DMSO-d₆): δ 13.51 (s, 1H), 13.45 (s, 1H), 10.30 (s, 1H), 9.59 (s, 1H), 8.79 (d, 1H), 8.24 (dd, 1H), 8.16 (d, 1H), 7.93 (s, 1H), 7.91 (s, 1H), 6.86 (s, 1H), 6.79 (s, 1H).

### Example 8

### N-(5-Fluoropyridin-2-yl)-pyrazole-3-carboxamide

A mixture of intermediate (III) (188 mg, 1.0 mmol), DMAP (122 mg, 1.0 mmol) and 2-amino-5-fluoropyridine (112 mg, 1.0 mmol) and DMF (5 mL) was stirred at 120° C. for 18 h. Water (10 mL) was added and the mixture was acidified to ˜pH 4 with HCl (aq., 2M) and extracted with EtOAc (15 mL). The combined extracts were washed with NaCl (sat., aq.), dried (MgSO₄) and concentrated. Crystallisation from EtOAc/toluene delivered the title compound as a white powder. Yield: 47 mg, (23%).

MS (M⁺+H) m/z=207

¹H-NMR (DMSO-d₆, 400 MHz), δ 13.53 (s, 1H), 9.76 (s, 1H), 8.36 (s, 1H), 8.19-8.23 (m, 1H), 7.93 (s, 1H), 7.84-7.77 (m, 1H), 6.86 (s, 1H)

### Examples 9-64

### General Procedures

### Method A

A mixture of the relevant substituted pyrazole-3-carboxylic acid (intermediate IV, V, VII or XI, 1.2 mmol) and SOCl₂ (10 mL) was stirred at 80° C. for 18 h. After cooling to rt the mixture was concentrated and the residue dried in vacuo. A mixture of the relevant arylamine (2.4 mmol), DMAP (1.6 mmol) and CH₂Cl₂ (10 mL) was added. The mixture was stirred at 60° C. for 18 h. After cooling to rt, the mixture was concentrated and HCl (aq., 1M; 10 mL) was added. The mixture was extracted with ethyl acetate (4×10 mL), the combined extracts washed with NaCl (sat., aq.; 20 mL), dried (Na₂SO₄) and concentrated. Crystallisation from ethanol/water (1:1) and EtOAc/hexane (2:1) gave the desired product.

### Method B

A mixture of the relevant dipyrazolo[1,5-a;1′,5′-d]pyrazine-4,9-dione (intermediate I or III, 1.5 mmol), the relevant arylamine (1.0 mmol), DMAP (12 mg, 0.10 mmol), DMF (0.5 mL) and pyridine (5 mL) was stirred at 80° C. for 18 h. After cooling to rt the mixture was concentrated and purified by chromatography (EtOAc/heptane) to give the desired compound.

### Method C

A mixture of the relevant substituted pyrazole-3-carboxylic acid (intermediate IV or IX, 1.0 mmol) and SOCl₂ (0.70 mL, 10 mmol) in 1,2-dichloroethane (5 mL) was heated at reflux for 18 h. After cooling to rt the mixture was concentrated and the residue was dried in vacuo. A mixture of the relevant arylamine (1.0 mmol), DMAP (12 mg, 0.10 mmol), DMF (0.5 mL) and pyridine (1 mL) was added. The mixture was stirred at 80° C. for 18 h. After cooling to rt the mixture was concentrated and purified by chromatography (EtOAc/heptane) to give the desired compound.

### Method D

Sodium hydride (60% in mineral oil, 60 mg, 1.5 mmol) was added to a solution of the relevant arylamine (1.0 mmol) in DMF (2 mL) at rt. The mixture was stirred for 5 mint then a solution of the relevant substituted pyrazole-3-carboxylic acid ester (intermediate VI, VIII or X, 0.50 mmol) in DMF (2 mL) was added and the mixture was stirred at rt for 15 h. The mixture was acidified with hydrochloric acid (1M, 10 mL) and extracted with EtOAc (3×20 mL). The combined organic phases were washed with brine (20 mL), dried over anh. Na₂SO₄ and concentrated and purified by chromatography (EtOAc/heptane) to give the desired compound.

### Method E

A mixture of TBTU (642 mg, 2.0 mmol), pyrazole-3-carboxylic acid or 5-chloro pyrazole-3-carboxylic acid (intermediate II) (1.0 mmol), the relevant arylamine (1.0 mmol), DIPEA (348 μL, 2.0 mmol) and DMAP (12 mg, 0.1 mmol) in dry DMF (5 mL) was stirred at 80° C. for 3 days. After cooling to rt the mixture was concentrated and hydrochloric acid (1M, 10 mL) was added. The mixture was extracted with EtOAc (4×10 mL), the combined organic phases washed with NaCl (sat., aq.; 20 mL), dried (Na₂SO₄), concentrated and purified by chromatography (EtOAc/heptane) to give the desired compound.

### Method F

Oxalyl chloride (192 μL, 2.2 mmol) was added dropwise to a mixture of pyrazole-3-carboxylic acid or 5-chloro pyrazole-3-carboxylic acid (intermediate II) (2.0 mmol) and dry DMF (3 drops) in dry THF (10 mL) under argon at 0° C. The mixture was stirred at rt for 4 h, then DIPEA (1.12 mL, 6.4 mmol) followed by the relevant arylamine (2.2 mmol) were added. The mixture was stirred at 60° C. for 18 h. After cooling to rt, the mixture was concentrated and purified by chromatography (EtOAc/heptane) to give the desired compound.

### Method G

A mixture of dipyrazolo[1,5-a;1′,5′-d]pyrazine-4,9-dione (intermediate I, 348 mg, 1.85 mmol), DMAP (113 mg, 0.93 mmol) and the relevant arylamine (0.93 mmol) in DMF (2 mL) was stirred at 120° C. for 32 h. After cooling to rt, water (50 mL) was added and the white precipitate was filtered off and washed with water. Crystallisation from MeOH gave the desired compound.

TABLE 1
Examples (Ex.) 9 to 64

| | | Prepared from (PCA) or | | | |
|---|---|---|---|---|---|
| | | intermediate I-X and | | | Yield |
| Ex | Chemical Name | arylamine) | | Method | % |
| 9 | 4,5-Dichloro-N-(5-fluoropyridin- | IV | 2-Amino-5- | A | 51 |
| | 2-yl)pyrazole-3-carboxamide | | fluoropyridine | | |
| 10 | 4,5-Dichloro-N-(5- | IV | 2-Amino-5- | C | 6 |
| | (trifluoromethyl)pyridin-2- | | trifluoromethyl- | | |
| | yl)pyrazole-3-carboxamide | | pyridine | | |
| 11 | 4-Chloro-N-(5-fluoropyridin-2- | V | 2-Amino-5- | A | 36 |
| | yl)-5-(trifluoromethyl)pyrazole- | | fluoropyridine | | |
| | 3-carboxamide | | | | |
| 12 | 4-Chloro-N-(5-chloropyridin-2- | V | 2-Amino-5- | A | 23 |
| | yl)-5-(trifluoromethyl)pyrazole- | | chloropyridine | | |
| | 3-carboxamide | | | | |
| 13 | 5-(Difluoromethyl)-N-(5- | VI | 2-Amino-5- | D | 8 |
| | fluoropyridin-2-yl)pyrazole-3- | | fluoropyridine | | |
| | carboxamide | | | | |
| 14 | 5-Chloro-N-(5-nitropyridin-2- | III | 2-Amino-5- | B | 40 |
| | yl)pyrazole-3-carboxamide | | nitropyridine | | |
| 15 | 5-Chloro-N-(5,6-dimethyl- | II | 2-Amino-5,6- | E | 41 |
| | pyridin-2-yl)pyrazole-3- | | dimethyl- | | |
| | carboxamide | | pyridine | | |
| 16 | 5-Chloro-N-(6-methoxypyridin- | III | 2-Amino-6- | B | 2 |
| | 2-yl)pyrazole-3-carboxamide | | methoxy- | | |
| | | | pyridine | | |
| 17 | N-(5-Bromo-3-methylpyridin-2- | II | 2-Amino-5- | E | 6 |
| | yl)-5-chloropyrazole-3- | | bromo-3- | | |
| | carboxamide | | methylpyridine | | |
| 18 | 5-Chloro-N-(5,6- | II | 2-Amino-5,6- | E | 29 |
| | dimethoxypyridin-2-yl)pyrazole- | | dimethoxy- | | |
| | 3-carboxamide | | pyridine | | |
| 19 | 5-Chloro-N-(3-methoxypyridin- | II | 2-Amino-3- | F | 26 |
| | 2-yl)pyrazole-3-carboxamide | | methoxy- | | |
| | | | pyridine | | |
| 20 | 5-Chloro-N-(5-ethylpyridin-2- | II | 2-Amino-5- | E | 47 |
| | yl)pyrazole-3-carboxamide | | ethylpyridine | | |
| 21 | Methyl 6-(5-chloropyrazole-3- | II | 6-Amino- | E | 2 |
| | carboxamido)nicotinate | | nicotinic acid | | |
| | | | methyl ester | | |
| 22 | 5-Chloro-N-(5-phenylpyridin-2- | II | 2-Amino-5- | E | 5 |
| | yl)pyrazole-3-carboxamide | | phenylpyridine | | |
| 23 | N-(5-butylpyridin-2-yl)-5- | II | 2-Amino-5- | E | 13 |
| | chloropyrazole-3-carboxamide | | butylpyridine | | |
| 24 | 5-Chloro-N-(5-ethoxypyridin-2- | III | 2-Amino-5- | B | 14 |
| | yl)pyrazole-3-carboxamide | | ethoxypyridine | | |
| 25 | 5-Chloro-N-(5-propoxypyridin-2- | II | 2-Amino-5- | E | 42 |
| | yl)pyrazole-3-carboxamide | | propoxypyridine | | |
| 26 | N-(6-Bromopyridin-2-yl)-5- | III | 2-Amino-6- | B | 32 |
| | chloropyrazole-3-carboxamide | | bromopyridme | | |
| 27 | N-(5-Bromopyridin-2-yl)-5- | III | 2-Amino-5- | B | 4 |
| | chloropyrazole-3-carboxamide | | bromopyridine | | |
| 28 | N-(5-Butoxypyridin-2-yl)-5- | III | 2-Amino-5- | B | 49 |
| | chloropyrazole-3-carboxamide | | butoxypyridine | | |
| 29 | 5-Chloro-N-(5-methoxypyridin- | III | 2-Amino-5- | B | 75 |
| | 2-yl)pyrazole-3-carboxamide | | methoxy- | | |
| | | | pyridine | | |
| 30 | 5-Chloro-N-(5-propylpyridin-2- | II | 2-Amino-5- | E | 26 |
| | yl)pyrazole-3-carboxamide | | propylpyridine | | |
| 31 | 5-Chloro-N-(5-ethyl-6- | II | 2-Amino-5- | E | 60 |
| | methylpyridin-2-yl)pyrazole-3- | | ethyl-6- | | |
| | carboxamide | | methylpyridine | | |
| 32 | 5-Chloro-N-(5- | II | 2-Amino-5-(tert- | E | 9 |
| | (hydroxymethyl)pyridin-2- | | butyl- | | |
| | yl)pyrazole-3-carboxamide | | dimethylsilyl- | | |
| | | | oxymethyl)- | | |
| | | | pyridine | | |
| | | | hydrochloride | | |
| 33 | 4-Chloro-N-(5-fluoropyridin-2- | VII | 2-Amino-5- | A | 33 |
| | yl)pyrazole-3-carboxamide | | fluoropyridine | | |
| 34 | N-(5-Fluoropyridin-2-yl)-5- | VIII | 2-Amino-5- | D | 24 |
| | methyl-4-(trifluoromethyl)- | | fluoropyridine | | |
| | pyrazole-3-carboxamide | | | | |
| 35 | N-(5-Fluoropyridin-2-yl)-4,5- | IX | 2-Amino-5- | C | 11 |
| | bis(trifluoromethyl)pyrazole-3- | | fluoropyridine | | |
| | carboxamide | | | | |
| 36 | 4-Chloro-N-(5-fluoropyridin-2- | X | 2-Amino-5- | D | 34 |
| | yl)-5-methylpyrazole-3- | | fluoropyridine | | |
| | carboxamide | | | | |
| 37 | N-(5-Ethylpyridin-2-yl)pyrazole- | I | 2-Amino-5- | B | 67 |
| | 3-carboxamide | | ethylpyridine | | |
| 38 | N-(5,6-Dimethylpyridin-2- | PCA | 2-Amino-5,6- | E | 30 |
| | yl)pyrazole-3-carboxamide | | dimethyl- | | |
| | | | pyridine | | |
| 39 | N-(5-Nitropyridin-2-yl)pyrazole- | PCA | 2-Amino-5- | F | 0.5 |
| | 3-carboxamide | | nitropyridine | | |
| 40 | N-(3-Chloro-5- | I | 2-Amino-3- | G | 20 |
| | trifluoromethylpyridin-2- | | chloro-5- | | |
| | yl)pyrazole-3-carboxamide | | trifluoromethyl- | | |
| | | | pyridine | | |
| 41 | N-(6-Trifluoromethylpyridin-2- | I | 2-Amino-6- | G | 21 |
| | yl)pyrazole-3-carboxamide | | trifluoromethyl- | | |
| | | | pyridine | | |
| 42 | N-(6-Methoxypyridin-2- | I | 2-Amino-6- | B | 24 |
| | yl)pyrazole-3-carboxamide | | methoxy- | | |
| | | | pyridine | | |
| 43 | N-(5-Bromopyridin-2- | I | 2-Amino-5- | B | 2 |
| | yl)pyrazole-3-carboxamide | | bromopyridine | | |
| 44 | N-Methylpyridin-2- | PCA | 2-Amino-4- | E | 21 |
| | yl)pyrazole-3-carboxamide | | methylpyridine | | |
| 45 | N-Ethyl-6-methylpyridin-2- | I | 2-Amino-5- | B | 55 |
| | yl)pyrazole-3-carboxamide | | ethyl-6- | | |
| | | | methylpyridine | | |
| 46 | N-(5-Iodopyridin-2-yl)pyrazole- | I | 2-Amino-5- | B | 59 |
| | 3-carboxamide | | iodopyridine | | |
| 47 | N-(5-Methylpyridin-2- | PCA | 2-Amino-5- | E | 43 |
| | yl)pyrazole-3-carboxamide | | methylpyridine | | |
| 48 | N-(5-Methoxypyridin-2- | I | 2-Amino-5- | B | 35 |
| | yl)pyrazole-3-carboxamide | | methoxy- | | |
| | | | pyridine | | |
| 49 | N-(5,6-Dimethoxypyridin-2- | I | 2-Amino-5,6- | B | 40 |
| | yl)pyrazole-3-carboxamide | | dimethoxy- | | |
| | | | pyridine | | |
| 50 | N-(5-Bromo-4-methylpyridin-2- | I | 2-Amino-5- | B | 25 |
| | yl)pyrazole-3-carboxamide | | bromo-4- | | |
| | | | methylpyridine | | |
| 51 | N-(5-Ethoxypyridin-2- | I | 2-Amino-5- | B | 30 |
| | yl)pyrazole-3-carboxamide | | ethoxypyridine | | |
| 52 | N-(5-Propylpyridin-2- | I | 2-Amino-5- | B | 3 |
| | yl)pyrazole-3-carboxamide | | propylpyridine | | |
| 53 | N-(6-Methylpyridin-2- | I | 2-Amino-6- | G | 47 |
| | yl)pyrazole-3-carboxamide | | methylpyridine | | |
| 54 | Methyl 6-(pyrazole-3- | I | 6-Amino- | B | 29 |
| | carboxamido)nicotinate | | nicotinic acid | | |
| | | | methyl ester | | |
| 55 | N-(5-Bromo-3-methylpyridin-2- | I | 2-Amino-5- | B | 53 |
| | yl)pyrazole-3-carboxamide | | bromo-3- | | |
| | | | methylpyridine | | |
| 56 | N-(5-Phenylpyridin-2- | PCA | 2-Amino-5- | E | 9 |
| | yl)pyrazole-3-carboxamide | | phenylpyridine | | |
| 57 | N-(4,6-Dimethylpyridin-2- | PCA | 2-Amino-4,6- | E | 41 |
| | yl)pyrazole-3-carboxamide | | dimethyl- | | |
| | | | pyridine | | |
| 58 | N-(3-Methoxypyridin-2- | PCA | 2-Amino-3- | F | 4 |
| | yl)pyrazole-3-carboxamide | | methoxy- | | |
| | | | pyridine | | |
| 59 | N-(6-Bromopyridin-2- | I | 2-Amino-6- | B | 36 |
| | yl)pyrazole-3-carboxamide | | bromopyridine | | |
| 60 | N-(5-Cyanopyridin-2- | I | 2-Amino-5- | B | 3 |
| | yl)pyrazole-3-carboxamide | | cyanopyridine | | |
| 61 | N-(5-Butylpyridin-2-yl)pyrazole- | I | 2-Amino-5- | B | 33 |
| | 3-carboxamide | | butylpyridine | | |
| 62 | N-(5-Benzamidopyridin-2- | I | N-(6-Amino- | B | 43 |
| | yl)pyrazole-3-carboxamide | | pyridin-3-yl)- | | |
| | | | benzamide | | |
| | | | hydrochloride | | |
| 63 | N-(5-Propoxypyridin-2- | I | 2-Amino-5- | B | 44 |
| | yl)pyrazole-3-carboxamide | | propoxypyridine | | |
| 64 | 4-Chloro-5-difluoromethyl-N-(5- | XI | 2-Amino-5- | A | 5 |
| | fluoropyridin-2-yl)pyrazole-3- | | fluoropyridine | | |
| | carboxamide | | | | |

TABLE 2
Physical properties of the compounds of Examples 9-64

| | | MS | |
|---|---|---|---|
| | | (M⁻ − H), | |
| Ex. | M.W. | m/z | ^{¹}H NMR (DMSO-d₆, 400 MHz), δ |
| 9 | 275.07 | 273 | 14.43 (br. s, 1H), 10.49 (br. s, 1H), 8.41 (d, 1H), |
| | | | 8.14 (dd, 1H), 7.81 (ddd, 1H). |
| 10 | 325.07 | 323 | 14.53 (s, 1H), 10.83 (s, 1H), 8.81 (s, 1H), 8.35-8.27 |
| | | | (m, 2H) |
| 11 | 308.62 | 307 | 14.95 (br. s, 1H), 10.77 (br. s, 1H), 8.42 (d, 1H), |
| | | | 8.15 (dd, 1H), 7.82 (ddd, 1H) |
| 12 | 325.07 | 323 | 14.96 (br. s, 1H), 10.80 (br. s, 1H), 8.46 (s, 1H), |
| | | | 8.15 (d, 1H), 8.03-8.00 (m, 1H) |
| 13 | 256.18 | 255 | 14.30 (s, 1H), 11.13 (br. s, 1H), 8.43 (d, 1H), 8.21 |
| | | | (dd, 1H), 7.83 (dt, 1H), 7.53 (br. s, 1H), 7.09 (t, 1H) |
| 14 | 267.63 | 266 | 14.29 (br. s, 1H), 11.82 (br. s, 1H), 9.31 (d, 1H), |
| | | | 8.75 (dd, 1H), 8.48 (d, 1H), 7.47 (s, 1H) |
| 15 | 250.68 | 249 | 14.04-13.88 (br. s, 1H), 10.89-10.66 (br. s, 1H), |
| | | | 7.89 (d, 1H), 7.57 (d, 1H), 7.32 (s, 1H), 2.41 (s, |
| | | | 3H), 2.23 (s, 3H) |
| 16 | 252.66 | 251 | 14.04 (br. s, 1H), 10.56 (br. s, 1H), 7.80-7.72 (m, |
| | | | 2H), 7.32 (s, 1H), 6.62 (d, 1H), 3.89 (s, 3H) |
| 17 | 315.55 | 313 | 14.09-14.00 (br. s, 1H), 10.82-10.65 (br. s, 1H), |
| | | | 8.47 (d, 1H), 8.07 (d, 1H), 7.11 (s, 1H), 2.20 (s, 3H) |
| 18 | 282.68 | 281 | 13.98 (br. s, 1H), 10.41 (br. s, 1H), 7.63 (d, 1H), |
| | | | 7.38 (d, 1H), 7.27 (br. s, 1H), 3.91 (s, 3H), 3,78 (s, |
| | | | 3H) |
| 19 | 252.66 | 251 | 13.98 (s, 1H), 10.41 (s, 1H), 8.05 (dd, 1H), 7.57 (d, |
| | | | 1H), 7.36 (dd, 1H), 7.07 (s, 1H), 3.82 (s, 3H) |
| 20 | 250.68 | 249 | 14.09-13.93 (br. s, 1H), 10.96-10.76 (s, 1H), 8.27 |
| | | | (d, 1H), 8.08 (s, 1H), 7.73 (dd, 1H), 7.30 (s, 1H), |
| | | | 2.62(q, 2H), 1.20 (t, 3H) |
| 21 | 280.67 | 279 | 14.14 (s, 1H), 11.36 (br. s, 1H), 8.92 (d, 1H), 8.36 |
| | | | (dd, 1H), 8.33 (d, 1H), 7.38 (s, 1H), 3.88 (s, 3H). |
| 22 | 298.73 • | 297 | 14.07 (br. s, 1H), 11.07 (br. s, 1H), 8.74. (s, 1H), |
| | | | 8.30-8.16 (m, 2H), 7.80-7.72 (m, 2H), 7.51 (t, 2H), |
| | | | 7.46-7.34(m, 2H) |
| 23 | 278.74 | 277 | 14.02 (br. s, 1H), 10.86 (br. s, 1H), 8.24 (d, 1H), |
| | | | 6.08 (d, 1H), 7.71 (dd, 1H), 7.31 (br. s, 1H). 2.59 (t, |
| | | | 1H), 1.54-1.60 (m, 2H), 1.34-1.30 (m, 2H), 0.91 (t, |
| | | | 3H) |
| 24 | 266.68 | 265 | 14.00 (br. s, 1H), 10.79 (br. s, 1H), 8.11-8.06 (m, |
| | | | 2H), 7.50 (dd, 1H), 7.28 (s, 1H), 4.11 (q, 2H), 1.35 |
| | | | (t, 3H) |
| 25 | 280.71 | 279 | (br. s, 1H), 10.81 (br. s, 1H), 8.11 (d, 1H), 8.07 (d, |
| | | | 1H), 7.50 (dd, 1H), 7.28 (br. s, 1H), 4.01 (t, 2H), |
| | | | 1.70-1.75 (m, 2H), 0.99 (t, 3H) |
| 26 | 301.53 | 299 | 14.08 (br. s, 1H), 11.25 (br. s, 1H), 8.20 (d, 1H), |
| | | | 7.82 (t, 1H), 7.44, (d, 1H), 7.37 (s, 1H) |
| 27 | 301.53 | 299 | 14.08 (br. s, 1H), 11.13 (br. s, 1H), 8.54 (d, 1H), |
| | | | 8.18-8.08 (m, 2H), 7.33 (s, 1H) |
| 28 | 294.74 | 293 | 14.00 (br. s, 1H), 10.83 (br. s, 1H), 8.12 (d, 1H), |
| | | | 8.07 (d, 1 H), 7.50 (dd, 1H), 7.29 (s, 1H), 4.05 (t, |
| | | | 2H), 1.70-1.75 (m, 2H), 1.42-1.47 (m, 2H), 0.94 (t, |
| | | | 3H) |
| 29 | 252.66 | 251 | 14.00 (br. s, 1H), 10.85 (br. s, 1H), 8.12 (d, 1H), |
| | | | 8.08 (d, 1H), 7.51 (dd, 1H), 7.29 (s, 1H), 3.84 (s, |
| | | | 3H) |
| 30 | 264.71 | 263 | 14.09-13.92 (br. s, 1H), 11.01-10.77 (s, 1H), 8.24 |
| | | | (d, 1H), 8.09 (s, 1H), 7.71 (dd, 1H), 7.31 (s, 1H), |
| | | | 2.56 (t, 2H), 1.57-1.63 (m, 2H), 0.90 (t, 3H) |
| 31 | 264.71 | 263 | 14.06-13.88 (br. s, 1H), 10.91-10.66 (s, 1H), 7.92 |
| | | | (d, 1H), 7.60 (d, 1H), 7.32 (s, 1H), 2.60 (q, 2H), |
| | | | 2.44 (s, 3H), 1.16 (t, 3H) |
| 32 | 252.66 | 251 | 14.17-13.90 (br. s, 1H), 11.14-10.74 (br. s, 1H), |
| | | | 8.35 (d, 1H), 8.13 (d, 1H), 7.80 (dd, 1H), 7.32 (s, |
| | | | 1H), 5.29 (t, 1H), 4.52 (d, 2H) |
| 33 | 240.62 | 241 | 13.83 (br. s, 1H), 9.86 (br. s, 1H), 8.37 (d, 1H), |
| | | (M⁺ + H) | 8.16 (dd, 1H), 7.79 (ddd, 1H) |
| 34 | 288.20 | 287 | 13.83 (s, 1H), 10.10 (s, 1H), 8.38 (d, 1H), 8.16 (dd, |
| | | | 1H), 7.81 (ddd, 1H), 2.41 (d, 3H) |
| 35 | 342.17 | 341 | 15.56-14.90 (br. s, 1H), 11.76-11.45 (br. s, 1H), |
| | | | 8.44 (d, 1H), 8.16 (dd, 1H), 7.87 (ddd, 1H) |
| 36 | 254.65 | 253 | 13.63 (br. s, 1H), 9.76 (br. s, 1H), 8.37 (d, 1H), |
| | | | 8.18 (dd, 1H), 7.81 (ddd, 1H), 2.26 (s, 3H) |
| 37 | 216.24 | 215 | 13.51 (br. s, 1H), 9.520 (s, 1H), 8.21 (s, 1H), 8.10 |
| | | | (d, 1H), 7.93(d, 1H), 7.71 (dd, 1H), 2.60 (q, 2H), |
| | | | 1.19 (t, 3H) |
| 38 | 216.24 | 215 | 13.50 (br. s, 1H), 9.38 (s, 1H), 7.93-7.92 (m, 2H), |
| | | | 7.57 (d, 1H), 6.84 (s, 1H), 2.37 (s, 3H), 2.22 (s, 3H) |
| 39 | 233.18 | 232 | 13.85-13.44 (br. s, 1H), 10.50-10.17 (br. s, 1H), |
| | | | 9.21 (d, 1H), 8.68 (dd, 1H), 8.41 (d, 1H), 7.96 (br. |
| | | | s, 1H), 6.95 (br. s, 1H) |
| 40 | 290.63 | 291 | 13.54 (br. s, 1H), 10.43 (br. s, 1H), 8.85 (s, 1H), |
| | | (M⁺ + H) | 8.56 (s, 1H), 7.95 (s, 1H), 6.84 (br. s, 1H) |
| 41 | 256.18 | 257 | 13.60 (br. s, 1H), 9.96 (br. s, 1H), 8.47-8.42 (m, |
| | | (M⁺ + H) | 1H), 8.16-8.12 (m, 1H), 7.95 (s, 1H), 7.67-7.64 (m, |
| | | | 1H), 6.92 (br. s, 1H) |
| 42 | 218.21 | 217 | 13.56 (br. s, 1H), 9.35 (s, 1H), 7.93 (s, 1H), 7.78- |
| | | | 7.70 (m, 2H), 6.86 (s, 1H), 6.61-6.53 (m, 1H), 3.86 |
| | | | (s, 3H) |
| 43 | 267.08 | 265 | 13.58 (br. s, 1H), 9.83 (br. s, 1H), 8.49 (d, 1H), |
| | | | 8.16 (d, 1H), 8.10 (dd, 1H), 7.90 (br. s, 1H), 6.92 |
| | | | (br. s, 1H) |
| 44 | 202.21 | 201 | 13.70-13.38 (br s, 1H), 9.66-9.37 (br. s, 1H), 8.20 |
| | | | (d, 1H), 8.04 (s, 1H), 7.99-7.86 (br. s, 1H), 7.00 (d, |
| | | | 1H), 6.94-6.79 (br. s, 1 H), 2.36 (s, 3H) |
| 45 | 230.27 | 229 | 13.5 (br. s, 1H), 9.28 (br. s, 1H), 7.96-7.90 (m, 2H), |
| | | | 7.58 (d, 1H), 6.84 (s, 1H), 2.58 (q, 2H), 2.41 (s, |
| | | | 3H), 1.16 (t, 3H) |
| 46 | 314.08 | 313 | 13.86-13.35 (br. s, 1H), 9.94-9.59 (br. s, 1H), 8.58 |
| | | | (d, 1H), 8.19 (dd, 1H), 8.06 (d, 1H), 7.98-7.79 (br. |
| | | | s, 1H), 7.02-6.82 (br. s, 1H) |
| 47 | 202.21 | 201 | 13.70-13.38 (br. s, 1H), 9.66-9.37 (br. s, 1H), 8.20 |
| | | | (d, 1H), 8.04 (s, 1H), 7.99-7.86 (br. s, 1H), 7.00 (d, |
| | | | 1H), 6.94-6.79 (br. s, 1 H), 2.36 (s, 3H) |
| 48 | 218.21 | 217 | 13.51 (br. s, 1H), 9.53 (br. s, 1H), 8.09 (d, 1H), |
| | | | 8.07 (d, 1H), 7.91 (br. s, 1H), 7.52 (dd, 1H), 6.85 |
| | | | (br. s, 1H), 3.82 (s 3H) |
| 49 | 248.24 | 247 | 13.48 (br. s, 1H), 9.22 (br. s, 1H), 7.92 (br. s, 1H), |
| | | | 7.65 (d, 1H), 7.39 (d, 1H), 6.81 (br. s, 1H), 3.88 (s, |
| | | | 1H), 3.77 (s, 1H) |
| 50 | 281.11 | 279 | 13.64-13.48 (br. s, 1H), 9.78-9.63 (br. s, 1H), 8.45 |
| | | | (s, 1H), 8.21 (s, 1H), 7.98-7.89 (br. s, 1H), 6.94- |
| | | | 6.83 (br. s, 1H), 2.41 (s, 3H) |
| 51 | 232.24 | 231 | 13.52 (br. s, 1H), 9.52 (br. s, 1H), 8.09 (d, 1H), |
| | | | 8.06 (d, 1H), 7.91 (br. s, 1H), 7.50 (dd, 1H), 6.86 |
| | | | (br. s, 1H), 4.11 (q, 2H), 1.34 (t, 3H) |
| 52 | 230.27 | 229 | 13.55 (br. s, 1H), 9.62 (br. s, 1H), 8.20 (d, 1H), |
| | | | 8.10 (d, 1H), 7.70 (dd, 1H), 6.91 (s, 1H), 2.54 (q, |
| | | | 1H, nearly hidden under solvent peak), 1.57-1.63 |
| | | | (m, 2H), 0.90 (t, 3H) |
| 53 | 202.21 | 201 | 13.70-13.45 (br. s, 1H), 9.62-9.33 (br. s, 1H), 7.99 |
| | | | (d, 1H), 7.98-7.81 (br. s, 1H), 7.73 (dd, 1H), 7.02 |
| | | | (d, 1H), 6.97-6.79 (br. s, 1H), 2.43 (s, 3H) |
| 54 | 246.22 | 245 | 13.88-13.34 (br. s, 1H), 10.37-9.83 (br. s, 1H), 8.89 |
| | | | (dd, 1H), 8.36 (dd, 1H), 8.33 (dd, 1H), 7.91 (s, 1H), |
| | | | 6.97 (s, 1H), 3.88 (s, 3H) |
| 55 | 281.11 | 279 | 13.88-13.10 (br. s, 1H), 10.55-9.77 (br. s, 1H), 8.42 |
| | | | (d, 1H), 8.02 (d, 1H), 7.93-7.77 (br. s, 1H), 6.92- |
| | | | 6.77 (br. s, 1H), 2.21 (s, 3H) |
| 56 | 264.28 | 263 | 13.62 (br. s, 1H), 9.78 (br. s, 1H), 8.70 (dd, 1H), |
| | | | 8.29 (dd, 1H), 8.18 (dd, 1H), 7.92 (br. s, 1H), 7.77- |
| | | | 7.71 (m, 2H), 7.46-7.55 (m, 2H), 7.36-7.44 (m, |
| | | | 1H), 6.95 (br. s 1H) |
| 57 | 216.24 | 215 | 13.72-13.39 (br. s, 1H), 9.54-9.28 (br. s, 1H), 8.01- |
| | | | 7.79 (br. s, 1H), 7.85 (s, 1H), 6.97-6.78 (br. s, 2H), |
| | | | 2.37 (s, 3H), 2.31 (s, 3H) |
| 58 | 218.21 | 217 | 13.64 (br. s, 0.35H), 13.4 (br. s, 0.65H), 10.40 (br. |
| | | | s, 0.35H), 9.65 (br. s, 0.65), 8.03 (d, 0.35H), 7.98 |
| | | | (d, 0.65 H), 7.91 (s, 1H), 7.75 (d, 0.35), 7.49 (d, |
| | | | 0.65H), 7.37-7.32 (m, 0.35H), 7.24-7.20 (m, |
| | | | 0.64H), 7.06 (s, 0.35H), 6.78 (s, 0.65H), 3.86 (s, |
| | | | 3H)¹ |
| 59 | 267.08 | 265 | 13.61 (br. s, 1H), 9.94 (br. s, 1H), 8.18 (d, 1H), |
| | | | 7.90 (br. s, 1H), 7.81 (t, 1H), 7.41 (d, 1H), 6.95 (br. |
| | | | s, 1H) |
| 60 | 213.20 | 212 | 13.63 (s, 1H), 10.11 (s, 1H), 8.84 (s, 1H), 8.33 (s, |
| | | | 2H), 7.96 (s, 1H), 6.92 (s, 1H) |
| 61 | 244.29 | 243 | 15.56 (br. s, 1H), 9.60 (br. s 1H), 8.20 (d, 1H), 8.10 |
| | | | (d, 1H), 7.89 (br. s, 1H), 7.70 (dd, 1H), 6.91 (br. s, |
| | | | 1H), 2.08 (t, 2H), 1.62-1.50 (m, 2H), 1.29-1.34 (m, |
| | | | 2H), 0.91 (t, 3H) |
| 62 | 307.31 | 306 | 13.63-13.44 (br. s, 1H), 10.45 (s, 1H), 9.68-9.59 |
| | | | (br. s, 1H), 8.76 (s, 1H), 8.21 (d, 1H), 8.20 (d, 1H), |
| | | | 8.01-7.97 (m, 2H), 7.95-7.89 (br. s, 1H), 7.64-7.50 |
| | | | (m, 3H), 6.93-6.84 (br. s, 1H) |
| 63 | 246.27 | 245 | 13.49 (s, 1H), 9.51 (s, 1H), 8.08 (d, 1H), 8.07 (d, |
| | | | 1H), 7.92 (s, 1H), 7.51 (dd, 1H), 6.84 (s, 1H), 4.00 |
| | | | (t, 2H), 1.70-1.75 (m, 2H), 0.99 (t, 3H) |
| 64 | 290.63 | 289 | 14.62 (br. s, 1H), 10.45 (s, 1H), 8.40 (d, 1H), 8.17 |
| | | | (dd, 1H), 7.84 (ddd, 1H), 7.18 (t, 1H) |

¹1:2 mixture of tautomers observed

### Example 65

### N-(5-Aminopyridin-2-yl)pyrazole-3-carboxamide

A mixture of 5-chloro-N-(5-nitropyridin-2-yl)pyrazole-3-carboxamide (Example 14, 50 mg, 0.18 mmol), Pd on carbon (10%, 20 mg) and DMF (1 mL) in CH₂Cl₂ (5 mL) was stirred under hydrogen atmosphere (atmospheric pressure) at rt for 15 h. The mixture was filtered through Celite® and the filtrate was concentrated in vacuo. The crude product was purified by column chromatography (EtOAc/heptane) to give the title compound as white solid. Yield 9 mg (24%).

¹H NMR (DMSO-d₆, 400 MHz) δ 13.44 (br. s, 1H), 9.24 (br. s, 1H), 7.89-7.95 (m, 2H), 7.03 (dd, 1H), 6.82 (br. s, 1H), 5.16 (s, 2H).

### Example 66

Title compounds of the Examples were tested in the biological test described above and were found to exhibit an IC₅₀ of 10 μM or below. For example, the following representative compounds of the examples exhibited the following IC₅₀ values:

### Example 5: 300 nM

### Example 6: 320 nM

### Example 8: 430 nM

### Example 11: 310 nM

### Example 13: 393 nM

### Example 14: 248 nM

### Example 29: 122 nM

Example 44: 346 nM

1. A compound of formula I, 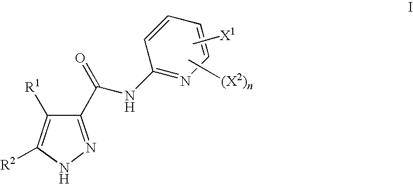
wherein
R¹ and R² independently represent H, halo or C₁₋₆ alkyl optionally substituted by one or more halo atoms;
X¹ represents H, halo or R^{3a};
X² represents:
1) G¹;
2) aryl or heteroaryl, both of which are optionally substituted by one or more substituents selected from A¹, —N₃, —NO₂ and —S(O)ₚR^{6e}; and
3) heterocycloalkyl, which is optionally substituted by one or more substituents selected from A², —N₃, —NO₂ and ═O;
G¹ represents halo, —R^{3a}, —CN, —C(O)R^{3b}, —C(O)OR^{3c}, —C(O)N(R^{4a})R^{5a}, —N(R^{4b})R^{5b}, —N(R^{3d})C(O)R^{4c}, —N(R^{3e})C(O)N(R^{4d})R^{5d}, —N(R^{3f})C(O)OR^{4e}, —N₃, —NO₂, —N(R^{3g})S(O)₂N(R^{4f})R^{5f}, —OR^{3h}, —OC(O)N(R^{4g})R^{5g}, —OS(O)₂R³ⁱ, —S(O)ₘR^{3j}, —N(R^{3k})S(O)₂R^{3m}, —OC(O)R³ⁿ, —OC(O)OR^{3p}, —S(O)₂N(R^{4h})R^{5h}, —S(O)₂OH, —P(O)(OR⁴ⁱ)(OR⁵ⁱ) or —C(O)N(R^{3q})S(O)₂R^{3r};
R^{3a} represents C₁₋₆ alkyl optionally substituted by one or more substituents selected from Z, F, Cl, —N(R^{6b})R^{6c}, —N₃, ═O and —OR^{6d};
R^{3b}, R^{3c}, R^{3h}, R³ⁿ, R^{4a} to R^{4h}, independently represent H, Z or C₁₋₆ alkyl optionally substituted by one or more halo atoms or —OR^{6d};
R^{3d} to R^{3g}, R^{3k}, R^{3q}, R^{5a}, R^{5b}, R^{5d} and R^{5f} to R^{5h} independently represent H or C₁₋₆ allyl optionally substituted by one or more halo atoms or —OR^{6d}; or
any of the pairs R^{4a} and R^{5a}, R^{4b} and R^{5b}, R^{4d} and R^{5d}, R^{4f} and R^{5f}, R^{4g} and R^{5g}, and R^{4h} and R^{5h}, may be linked together to form a 3- to 6-membered ring, which ring optionally contains a further heteroatom in addition to the nitrogen atom to which these substituents are necessarily attached, and which ring is optionally substituted by ═O or C₁₋₆ alkyl optionally substituted by one or more fluoro atoms;
R³ⁱ, R^{3j}, R^{3m}, R^{3p} and R^{3r} independently represent Z or C₁₋₆ alkyl optionally substituted by one or more substituents selected from B¹;
R⁴ⁱ and R⁵ⁱ independently represent H or C₁₋₆ alkyl optionally substituted by one or more substituents selected from B²;
Z represents:
a) heterocycloalkyl optionally substituted by one or more substituents selected from A³ and ═O;
b) aryl or heteroaryl both of which are optionally substituted by one or more substituents selected from A⁴, —N₃, —NO₂ and —S(O)_{q}R^{7e};
A¹, A², A³ and A⁴ independently represent halo, —R^{6a}, —CN, —N(R^{6b})R^{6c} or —OR^{6d};
R^{6b} to R^{6d} independently represent H or C₁₋₆ alkyl optionally substituted by one or more substituents selected from B³;
R^{6a}, R^{6e} and R^{7e} independently represent C₁₋₆ alkyl optionally substituted by one or more substituents selected from B⁴; or
R^{6b} and R^{6c} may be linked together to form a 3- to 6-membered ring, which ring optionally contains a further heteroatom in addition to the nitrogen atom to which these substituents are necessarily attached, and which ring is optionally substituted by ═O or C₁₋₆ alkyl optionally substituted by one or more fluoro atoms;
B¹, B², B³ and B⁴ independently represent F, Cl, —OCH₃, —OCH₂CH₃, —OCHF₂, —OCH₂CF₃, —OCF₃ or —OCF₂CF₃;
m, p and q independently represent 0, 1 or 2; and
n represents 0, 1, 2 or 3,
or a pharmaceutically-acceptable salt thereof,
provided that:
(A) when R¹ and R² both represent H and n represents 0, then X¹ does not represent a methyl substituent located at the 3- or 4-position of the 2-pyridyl ring,
(B) when R¹ represents H, R² represents methyl and n represents 0, then X¹ does not represent a methyl substituent located at the 4-position of the 2-pyridyl ring or a Br substituent located at the 5-position of the 2-pyridyl ring;
(C) when R¹ and R² both represent H and n represents 1, then X¹ and X² do not both represent methyl substituents located at the 4- and 6-positions of the 2-pyridyl ring.

2. A compound as claimed in claim 1, wherein X¹ represents Br, ethyl, butyl, propyl, hydroxymethyl, iodo, H, F, Cl, CH₃ or CF₃.

3. A compound as claimed in claim 2, wherein X¹ represents H, F, Cl, CH₃or CF₃.

4. A compound as claimed claim 1, wherein, when X² represents G¹, then G¹ represents Br, F, Cl, —R^{3a}, —CN, —C(O)N(R^{4a})R^{5a}, —N(R^{4b})R^{5b}, —N(R^{3d})C(O)R^{4c}, —OR^{3h}, —S(O)ₘR^{3j} or —S(O)₂N(R^{4h})R^{5h}.

5. A compound as claimed claim 1, wherein R^{3a} represents C₁₋₄ alkyl optionally substituted by one or more substituents selected from —OR^{6d} and fluoro.

6. A compound as claimed in claim 5, wherein R^{3a} represents C₁₋₄ alkyl optionally substituted by one or more fluoro atoms.

7. A compound as claimed in claim 1, wherein R^{3b}, R^{3c}, R^{3h} and R^{4d} to R^{4h} independently represent H or C₁₋₄ alkyl optionally substituted by one or more substituents selected from halo and —OR^{6d}.

8. A compound as claimed in claim 1, wherein R^{3d} to R^{3g} independently represent CH₃ or H.

9. A compound as claimed in claim 1, wherein R³ⁱ and R^{3j} independently represent C₁₋₄ alkyl optionally substituted by one or more F atoms.

10. A compound as claimed in claim 1, wherein R^{4a} to R^{4c} independently represent H, Z or C₁₋₄ alkyl optionally substituted by one or more substituents selected from halo and —OR^{6d}.

11. A compound as claimed in claim 1, wherein R^{5a}, R^{5b}, R^{5d} and R^{5f} to R⁵ⁱ independently represent H or C₁₋₄ alkyl optionally substituted by one or more substituents selected from halo and —OR^{6d} or R^{4b} and R^{5a}, R^{4b} and R^{5b}, R^{4d} and R^{5d}, R^{4f} and R^{5f}, R^{4g} and R^{5g}, and R^{4h} and R^{5h}, are linked together.

12. A compound of formula I as claim 1 but without the provisos, or a pharmaceutically acceptable salt thereof, for use as a pharmaceutical.

13. A pharmaceutical formulation including a compound of formula I, as defined in claim 1 without the provisos, or a pharmaceutically acceptable salt thereof, in admixture with a pharmaceutically acceptable adjuvant, diluent or carrier.

14. (canceled)

15. A method as claimed in claim 18 wherein the lipoxygenase is 15-lipoxygenase.

16. A method as claimed in claim 15, wherein the disease is inflammation and/or has an inflammatory component.

17. A method as claimed in claim 16 wherein the inflammatory disease is asthma, chronic obstructive pulmonary disease, pulmonary fibrosis, an allergic disorder, rhinitis, inflammatory bowel disease, an ulcer, inflammatory pain, fever, atherosclerosis, coronary artery disease, vasculitis, pancreatitis, arthritis, osteoarthritis, rheumatoid arthritis, conjunctivitis, iritis, scleritis, uveitis, a wound, dermatitis, eczema, psoriasis, stroke, diabetes, autoimmune diseases, Alzheimer's disease, multiple sclerosis, sarcoidosis, Hodgkin's disease or another malignancy.

18. A method of treatment of a disease in which inhibition of the activity of a lipoxygenase is desired and/or required, which method comprises administration of a therapeutically effective amount of a compound of formula I as defined in claim 1 but without the provisos, or a pharmaceutically-acceptable salt thereof, to a patient suffering from, or susceptible to, such a condition.

19. A combination product comprising:
(A) a compound of formula I as defined in claim 1 but without the provisos, or a pharmaceutically-acceptable salt thereof; and
(B) another therapeutic agent that is useful in the treatment of inflammation, wherein each of components (A) and (B) is formulated in admixture with a pharmaceutically-acceptable adjuvant, diluent or carrier.

20. A combination product as claimed in claim 19 which comprises a pharmaceutical formulation including a compound of formula I as defined in claim 1 but without the provisos, or a pharmaceutically-acceptable salt thereof, another therapeutic agent that is useful in the treatment of inflammation, and a pharmaceutically-acceptable adjuvant, diluent or carrier.

21. A combination product as claimed in claim 19 which comprises a kit of parts comprising components:
(a) a pharmaceutical formulation including a compound of formula I as defined in claim 1 but without the provisos, or a pharmaceutically-acceptable salt thereof, in admixture with a pharmaceutically-acceptable adjuvant diluent or carrier; and
(b) a pharmaceutical formulation including another therapeutic agent, that is useful in the treatment of inflammation in admixture with a pharmaceutically-acceptable adjuvant, diluent or carrier,
which components (a) and (b) are each provided in a form that is suitable for administration in conjunction with the other.

22. A process for the preparation of a compound of formula I as defined in claim 1, which comprises:
(i) for compounds of formula I in which R² represents halo or C₁₋₆ alkyl, reaction of a corresponding compound of formula I in which R² represents hydrogen, with an appropriate base followed by quenching with an appropriate electrophile;
(ii) for compounds of formula I in which R² represents CF₃, reaction of a corresponding compound of formula I in which R² represents bromo or iodo with CuCF₃ (or a source of CuCF₃);
(iii) reaction of a compound of formula III, 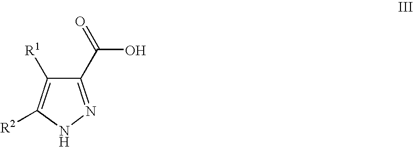
or a N-protected and/or O-protected derivative thereof, wherein R¹ and R² are as defined in claim 1, with a compound of formula IV, 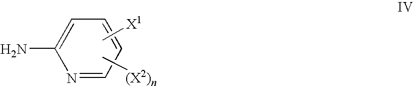
wherein X¹, X² and n are as defined in claim 1;
(iv) reaction of a compound of formula V, 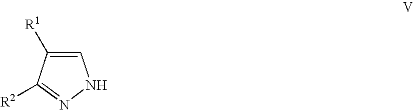
wherein R¹ and R² are as defined in claim 1, with a suitable base, followed by reaction with a compound of formula VI, 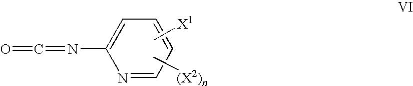
wherein X¹, X² and n are as defined in claim 1, followed by quenching with a suitable proton source;
(v) for compounds of formula I in which R² represents hydrogen and R¹ is as defined in claim 1, removal of the group J from a compound of formula VII, 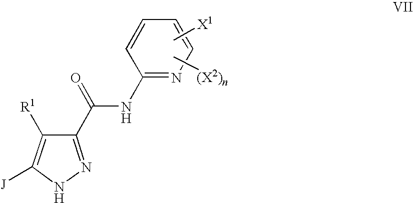
wherein J represents —Si(R^{t})₃ or —Sn(R^{z})₃ (in which each R^{t} independently represents a C₁₋₆ alkyl group or an aryl group and each R^{z} independently represents C₁₋₆ alkyl), and R¹, X¹, X² and n are as defined in claim 1;
(vi) reaction of a compound of formula VIII, 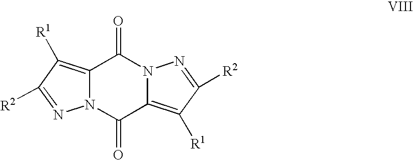
wherein R¹ and R² are as defined in claim 1, with a compound of formula IV as defined above;
(vii) for compounds of formula I in which one of R¹ or R² represents halo or C₁₋₆ alkyl optionally substituted as defined in claim 1, and the other represents H, reaction of a corresponding compound of formula I in which one of R¹ or R² represents bromo or iodo and the other represents H with a suitable organolithium base, followed by quenching with an appropriate electrophile; or
(viii) reaction of a compound of formula VIIIA 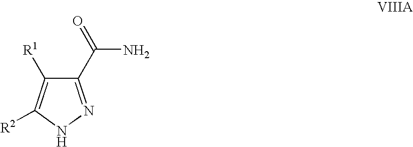
or a N-protected derivative thereof, wherein R¹ and R² are as defined in claim 1, with a compound of formula VIIIB, 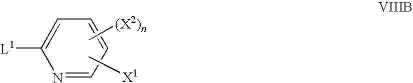
wherein L¹ represents a suitable leaving group, and X¹, X² and n are as defined in claim 1.

23. A process for the preparation of a pharmaceutical formulation as defined in claim 13, which process comprises bringing into association a compound of formula I, as defined in claim 1 but without the provisos, or a pharmaceutically acceptable salt thereof with a pharmaceutically-acceptable adjuvant, diluent or carrier.

24. A process for the preparation of a combination product as defined in claim 19, which process comprises bringing into association a compound of formula I, as defined in claim 1 but without the provisos, or a pharmaceutically acceptable salt thereof with the other therapeutic agent that is useful in the treatment of inflammation, and at least one pharmaceutically-acceptable adjuvant, diluent or carrier.
